(19) United States
(12) Patent Application Publication
(10) Pub. No.: US 2004/0034083 A1
(43) Pub. Date: Feb. 19, 2004

(54) Combination therapy for the treatment of Parkinson's disease with cyclooxygenase-2 (COX2) inhibitor(s)

(76) Inventors: Diane T. Stephenson, Portage, MI (US); Peter C. Isakson, Morrison Township, NJ (US); Timothy J. Maziasz, Northbrook, IL (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/413,348

(22) Filed: Apr. 14, 2003

Related U.S. Application Data
(60) Provisional application No. 60/373,311, filed on Apr. 18, 2002.

(51) Int. Cl. A61K 31/415; A61K 31/365; A61K 31/353

(52) U.S. Cl. 514/406; 514/456; 514/471

(57) ABSTRACT

A method of treating, preventing, or inhibiting PD, in a subject in need of such treatment, inhibition or prevention. The method comprises treating the subject with one or more cyclooxygenase-2 selective inhibitor(s) or isomer(s) or pharmaceutically acceptable salt(s), ester(s), or prodrug(s) thereof, in combination with one or more second drugs, wherein the amount of the cyclooxygenase-2 selective inhibitor(s) or isomer(s) or pharmaceutically acceptable salt(s), ester(s), or prodrug(s) thereof in combination with the amount of second drug(s) constitutes a PD treatment, inhibition or prevention effective amount.

This application claims benefit of provisional application No. 60/373,311, filed Apr. 18, 2002.

### BACKGROUND OF THE INVENTION

(1) Field of the Invention

The present invention relates to methods for the treatment of Parkinson's disease. More particularly, the present invention is directed to methods for the treatment of Parkinson's disease with cyclooxygenase-2 (COX 2) inhibitor(s) and second drug(s) in combination therewith.

(2) Description of Related Art

Parkinson's disease (PD) is a serious neurodegenerative disorder afflicting millions of people world-wide. It is believed that more than 1% of the population over 65 years of age is afflicted with PD. Standaert et al., Update on the Management of Parkinson's Disease, Contemporary Clinical Neurology, Vol. 77, No. 1, pp. 169-183 (January 1993). Prevalent PD related symptoms include resting tremors (e.g., shaking or 4-8 Hz pill rolling tremor of one hand which is maximal at rest, diminishes during movement and is absent during sleep; trembling on one side or both sides of the body in the hands, arms, legs, jaw, and face), rigidity (muscle stiffness; “ratchet” type resistance to classic movement), bradykinesia (a reduction in the amount of spontaneous movement, loss of normal movement and/or slow initiation of voluntary movement), and postural defects (inability to maintain an upright posture of the trunk, especially while standing or walking often manifested as as a stooped postural position together with a gait). Additional signs of PD include reduced blinking, microphonia (a lowered voice volume characterized by speaking softly in monotone voice), micrographia (typically, reduced writing width size with increase in vertical character height manifested as small, cramped, spidery handwriting), impaired ocular conversion, sialorrhea (excessive salivation), and/or seborrhea (abnormally facial oily appearance on the forehead), loss of facial expression, and freezing (especially when crossing a doorway), sleeping difficulties (inability/difficulty with changing position during sleep), swallowing difficulties, constipation, fatigue or general malaise, losing track of a word or thought, irritability or sadness for no apparent reason, lack of expression in the face, lack of animation, depression, hallucinations, senility, emotional changes, urinary problems, skin problems, among others.

Presently, PD therapy is limited to symptomatic relief of PD associated symptoms. Accordingly, such therapy does not arrest the continuing neurodegenerative nature of PD. Consequently, the symptoms of PD continue to worsen over time. Ultimately, at an advanced stage of PD, patients become bedridden, unable to eat, and tend to aspirate (inhale material into the respiratory tract) often. At such point, patients require full-time supportive care. The Merck Manual of Diagnosis and Therapy, M. H. Beers and R. Berkow, Eds., Seventeenth Edition, Publisher: Merck Research Laboratories, Whitehouse Station, N.J., pp. 1466-1470 (1999).

The neurological degenerative changes associated with PD include the gradual loss of dopaminergic neurons in the substantia nigra pars compacta, resulting in a continuing loss of dopaminergic terminals in the striatum. Thus, during the early stages of PD, when there is a lesser degree of neurodegeneration of dopaminergic neurons, PD responds better to symptomatic drug treatment. However, as PD progresses with increased loss of dopaminergic neurons, PD becomes more resistant to drug treatment requiring larger and/or more frequent dosing with drugs that yield an attenuated beneficial result for increasingly shorter periods of time. Often, prolonged treatment with higher and/or more frequent doses results in undesirable side effects from the drug treatment itself.

According to Lang, A. E., and Lozano, A. M., Parkinson's Disease, Review Article, Second of Two Parts, The New England Journal of Medicine, pp. 1130-1143 (Oct. 15, 1998), levodopa is the gold standard for the treatment of PD. For levodopa to be effective for the symptomatic treatment of PD, it must first cross the blood brain barrier (BBB) to reach the brain. There, the levodopa is converted to dopamine which provides symptomatic relief of PD. However, when levodopa alone is administered orally, only about 1% reaches the brain where it is converted to dopamine. Orally administered levodopa is metabolized by a decarboxylase enzyme into a metabolite form that does not easily cross the BBB. Up to 99% of orally administered levodopa is metabolized by decarboxylase and is then unable to cross the BBB. To increase the amount of levodopa that crosses the BBB into the brain, the decarboxylase metabolization of levodopa is blocked with a decarboxylase inhibitor known as carbidopa. Thus, when co-administered with carbidopa, a substantially increased amount of levodopa reaches the brain where levodopa is converted to dopamine, which counteracts the undesirable symptoms of PD. When co-administered with carbidopa, the beneficial effects of levodopa become more pronounced in combating the symptoms of PD.

However, levodopa's effectiveness typically lasts for about 5 years after initiation of therapy with levodopa/carbidopa therapy. Thereafter, continued use of levodopa is much less effective in the treatment of PD and its continued use is associated with numerous side effects. See Lang et al. at 1135, Table 3, listing various problems associated with prolonged levodopa PD therapy. These problems include early suboptimal symptom control, treatment resistant motor and non-motor symptoms, motor fluctuations, dyskinesias (abnormal involuntary movements), psychiatric disturbances and transient “on” and “off” episodes. Because the effectiveness of levodopa is limited to about 5 years of levodopa/carbidopa therapy in combating PD symptoms, it is desirable to delay the initiation of levodopa/carbidopa therapy to relieve the more severe PD associated symptoms present during the latter stages of PD. Thus, there is a need to find other drugs for treating PD.

Under certain conditions of chronic neurodegeneration, neuroinflammation may be observed. However, the functional consequences of chronic inflammatory processes in the brain are not well understood.

Recently, compounds that selectively inhibit cyclooxygenase-2 have been discovered. These COX 2 inhibiting compounds selectively inhibit the activity of COX 2 to a greater extent than the activity of cyclooxygenase-1 (COX 1). COX 1 has been shown to be a constitutively produced enzyme that is involved in many of the non-inflammatory regulatory functions associated with prostaglandins. COX 2, on the other hand, is an inducible enzyme having significant involvement in the inflammatory process. See, Needleman, P. et al., J. Rheumatol., 24, Suppl.49:6-8 (1997). See, Fu, J. Y., et al., J. Biol. Chem., 265(28):16737-40 (1990). The new COX 2-selective inhibitors are believed to offer advantages that include avoiding harmful side effects associated with the inhibition of COX 1.

Information on the identification and/or use of cyclooxygenase-2-selective inhibitors can be found in references such as: (1) Buttgereit, F. et al., Am. J Med., 110(3 Suppl. 1):13-9 (2001); (2) Osiri, M. et al, Arthritis Care Res., 12(5):351-62 (1999); (3) Buttar, N. S. et al., Mayo Clin. Proc., 75(10):1027-38 (2000); (4) Wollheim, F. A., Current Opin. Rheumatol., 13:193-201 (2001); (5) U.S. Pat. Nos. 5,434,178 (1,3,5-trisubstituted pyrazole compounds); (6) 5,476,944 (derivatives of cyclic phenolic thioethers); (7) 5,643,933 (substituted sulfonylphenylheterocycles); 5,859,257 (isoxazole compounds); (8) 5,932,598 (prodrugs of benzenesulfonamide-containing COX 2 inhibitors); (9) 6,156,781 (substituted pyrazolyl benzenesulfonamides); (10) 6,110,960 (for dihydrobenzopyran and related compounds), (11) 6,180,651 (includes disclosure of BMS-347070), (12) Hillson, J. L. et al., Expert Opin. Pharmacother., 1(5):1053-66 (2000), (for rofecoxib, Vioxx®, Merck & Co., Inc.); (13) Everts, B. et al., Clin. Rheumatol., 19(5):331-43 (2000), (for celecoxib, Celebrex®, Pharmacia Corporation, and rofecoxib); (14) Jamali, F., J. Pharm. Pharm. Sci., 4(1):1-6 (2001), (for celecoxib); (15) U.S. Pat. Nos. 5,521,207 and 5,760,068 (for substituted pyrazolyl benzenesulfonamides); (16) Davies, N. M. et al., Clinical Genetics, Abstr. at http://www.mmhc.com/cg/articles/CG0006/davies.html (for celecoxib, valdecoxib, parecoxib, deracoxib, and rofecoxib); (17) http://www.celebrex.com (for celecoxib); (18) http://www.docguide.com/dg.nsf/PrintPrint/F1F8DDD2D8B0094085256 98F00742187, May 9, 2001 (for etoricoxib, MK-663, Merck & Co., Inc.); (19) Saag, K. et al., Arch. Fam. Med., 9(10):1124-34 (2000), (for rofecoxib); and (20) International Patent Publication No. WO 00/24719 (for ABT 963, Abbott Laboratories).

Various U.S. patents and patent applications discuss the treatment of a number of neurodegenerative and other diseases which include the following: (21) U.S. Pat. Nos. 6,005,000, 6,262,073 B1, and 6,136,832 (use of certain compounds of the formula

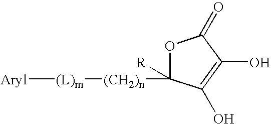

. . . ); (22) U.S. Pat. No. 6,063,807 (use of salt AB where A=a cyclooxygenase inhibitor,

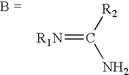

. . . ); (23) U.S. Pat. No. 6,277,878 B1 (use of

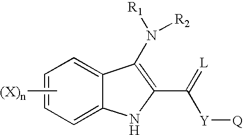

. . . ); (24) U.S. Pat. No. 6,303,613 B1 (use of

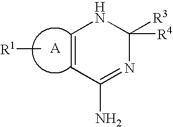

with celecoxib of MK 966 . . . ); (25) U.S. Pat. No. 6,303,628 B1 (use of

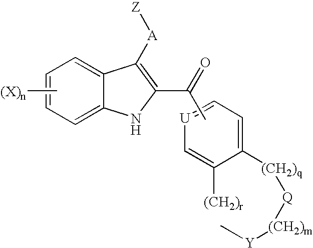

. . . ); (26) U.S. Pat. No. 6,306,842 (use of X-L-Y where

X=non-steroidal anti-inflammatory drug (NSAID),

L=an optional linker/spacer and

Y=a selective COX 2 inhibitor); (27) U.S. Pat. No. 6,147,080 (use of

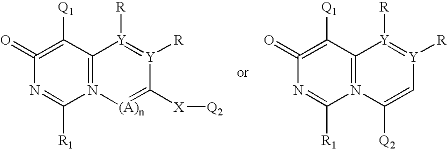

. . . ); (28) U.S. patent application Publication No. U.S. 2001/0025044 A1(use of compounds similar to those disclosed in 27); (29) U.S. Pat. No. 6,294,170 (use of celecoxib . . . ); and (30) U.S. Pat. No. 6,265,436 (use of

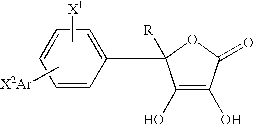

### SUMMARY OF THE INVENTION

According to one embodiment, the invention is directed to a novel method for the treatment, inhibition and/or prevention of PD (and/or its symptoms) comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, wherein said COX 2 inhibitor comprises a chromene that is a substituted benzopyran or is a chroman.

According to another embodiment, the invention is directed to a novel method for the treatment, inhibition and/or prevention of PD comprising administering, to a subject in need thereof, a therapeutically effective amount of cyclooxygenase-2 selective inhibitor which is I, II, III, IV, V, B-1, B-2, . . . B-231, or B-232 or combination(s) thereof (or an an isomer, a pharmaceutically acceptable salt, ester, or prodrug thereof, respectively), and a second drug which is C-1, C-2, C-3, C-4 . . . C-60, C-61, C-62, C-63, C-64, C-65, C-66, C-67, C-68, C-69, C-70, C-71, C-72, C-73, C-74, C-75, C-76, C-77, C-78, C-79, C-80, C-81 or C-82 or combination(s) thereof (or an isomer, a pharmaceutically acceptable salt, ester, or prodrug thereof, respectively). COX 2 inhibitors suitable for use with the present inventive method include, but are not limited to, those COX 2 inhibitors disclosed in Tables 1 and 1A below.

TABLE 1

| | |
|---|---|
| No. | Structure (COX 2 Inhibitor) |

| | | |
|---|---|---|
| I | 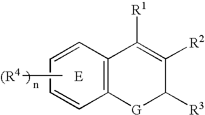 | (I) |
| | wherein R¹, R², R³, R⁴, | |
| | n and G are described herein. | |
| II | 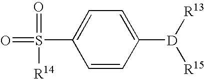 | (II) |
| | wherein R¹³, R¹⁴, R¹⁵, and | |
| | D are described herein. | |
| III | 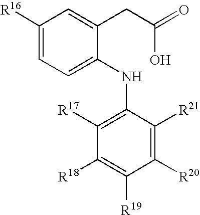 | (III) |
| | wherein R¹⁶, R¹⁷, R¹⁸, R¹⁹, R²⁰, and | |
| | R²¹, are as described herein. | |
| IV | 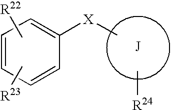 | (IV) |
| | wherein R²², R²³, R²⁴, X and J are | |
| | as described herein. | |
| V | 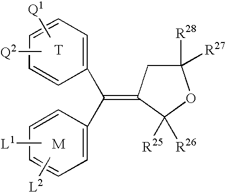 | (V) |
| | wherein Q¹, Q², L¹, L², R²⁵, R²⁶, | |
| | R²⁷ and R²⁸ are as described herein. | |

TABLE 1A

| First | |
|---|---|
| Drug | Name and/or Structure (COX 2 Inhibitor) |
| B-1 | 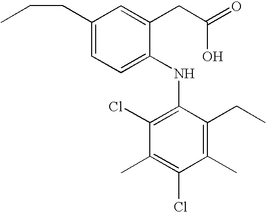 |
| | [2-(2,4-Dichloro-6-ethyl-3,5-dimethyl-phenylamino)- |
| | 5-propyl-phenyl]-acetic acid; |
| B-2 | 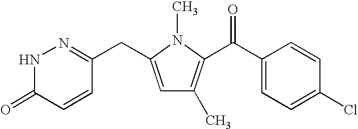 |
| | 6-[[5-(4-chlorobenzoyl)-1,4-dimethyl-1H-pyrrol-2-yl]methyl]- |
| | 3(2H)-pyridazinone or RS 57067; |
| B-3 | 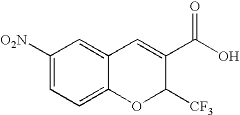 |
| | 6-Nitro-2-trifluoromethyl-2H-1- |
| | benzopyran-3-carboxylic acid; |
| B-4 | 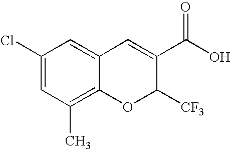 |
| | 6-Chloro-8-methyl-2-trifluoromethyl- |
| | 2H-1-benzopyran-3-carboxylic acid; |
| B-5 | 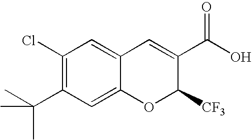 |
| | ((S)-6-Chloro-7-(1,1-dimethylethyl)-2-(trifluoromethyl- |
| | -2H-1-benzopyran-3-carboxylic acid; |
| B-6 | 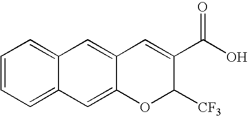 |
| | 2-Trifluoromethyl-2H-naphtho[2,3-b] |
| | pyran-3-carboxylic acid; |
| B-7 | 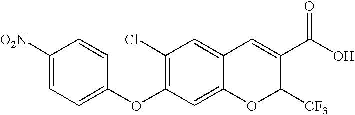 |
| | 6-Chloro-7-(4-nitrophenoxy)-2-(trifluoromethyl)-2H-1- |
| | benzopyran-3-carboxylic acid; |
| B-8 | 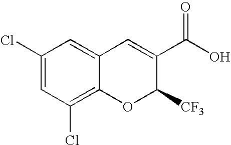 |
| | ((S)-6,8-Dichloro-2-(trifluoromethyl)- |
| | 2H-1-benzopyran-3-Carboxylic acid; |
| B-9 | 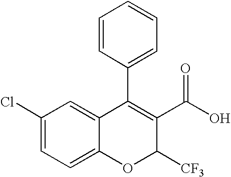 |
| | 6-Chloro-2-(trifluoromethyl)-4-phenyl-2H- |
| | 1-benzopyran-3-carboxylic acid; |
| B-10 | 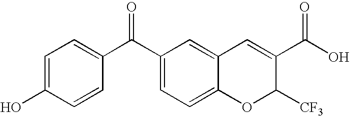 |
| | 6-(4-Hydroxybenzoyl)-2-(trifluoromethyl)- |
| | 2H-1-benzopyran-3-carboxylic acid; |
| B-11 | 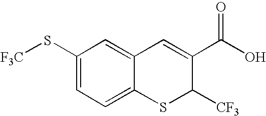 |
| | 2-(Trifluoromethyl)-6-[(trifluoramethyl)thio]- |
| | 2H-1-benzothiopyran-3-carboxylic acid; |
| B-12 | 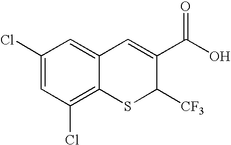 |
| | 6,8-Dichloro-2-trifluoromethyl-2H-1- |
| | benzothiopyran-3-carboxylic acid; |
| B-13 | 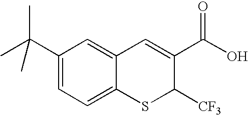 |
| | 6-(1,1-Dimethylethyl)-2-(trifluoromethyl)- |
| | 2H-1-benzothiopyran-3-carboxylic acid; |
| B-14 | 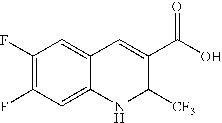 |
| | 6,7-Difluoro-1,2-dihydro-2-(trifluoromethyl)- |
| | 3-quinolinecarboxylic acid; |
| B-15 | 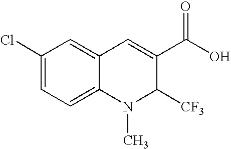 |
| | 6-Chloro-1,2-dihydro-1-methyl-2-(trifluoromethyl)- |
| | 3-quinolinecarboxylic acid; |
| B-16 | 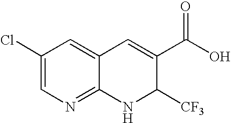 |
| | 6-Chloro-2-(trifluoromethyl)-1,2-dihydro |
| | [1,8]naphthyridine-3-carboxylic acid; |
| B-17 | 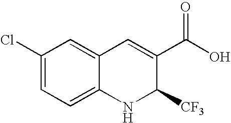 |
| | ((S)-6-Chloro-1,2-dihydro-2-(trifluoromethyl)- |
| | 3-quinolinecarboxylic acid |
| B-18 | 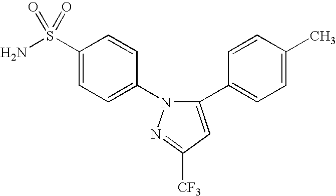 |
| | celecoxib; |
| B-19 | 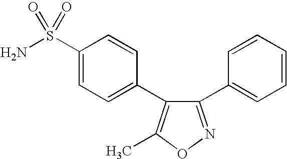 |
| | valdecoxib; |
| B-20 | 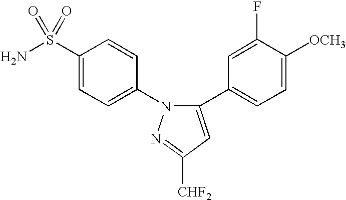 |
| | deracoxib; |
| B-21 | 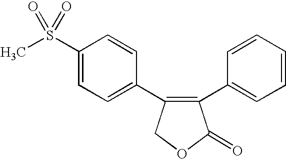 |
| | rofecoxib; |
| B-22 | 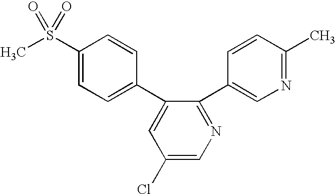 |
| | etoricoxib; |
| B-23 | 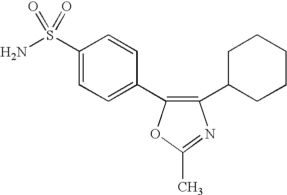 |
| | JTE-522; |
| B-24 | 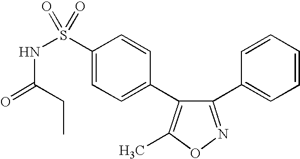 |
| | parecoxib; |
| B-25 | 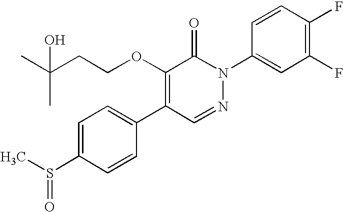 |
| | ABT-963 |
| B-26 | 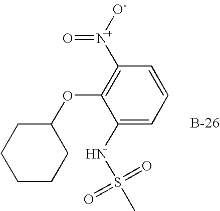 |
| | N-(2-cyclohexyloxynitrophenyl) methane sulfonamide or |
| | NS-398; |
| B-27 | 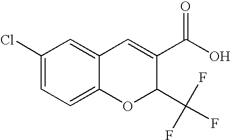 |
| | 6-chloro-2-trifluoromethyl-2H-1-benzopyran-3- |
| | carboxylic acid; |
| B-28 | 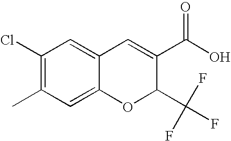 |
| | 6-chloro-7-methyl-2-trifluoromethyl-2H-1-benzopyran-3- |
| | carboxylic acid; |
| B-29 | 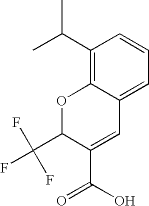 |
| | 8-(1-methylethyl)-2-trifluoromethyl-2H-1-benzopyran-3- |
| | carboxylic acid; |
| B-30 | 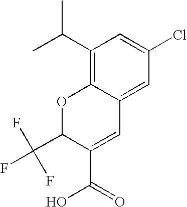 |
| | 6-chloro-8-(1-methylethyl)-2-trifluoromethyl-2H-1- |
| | benzopyran-3-carboxylic acid; |
| B-31 | 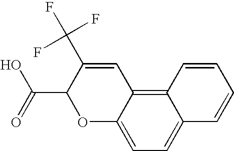 |
| | 2-trifluoromethyl-3H-naphtho[2,1-b]pyran-3- |
| | carboxylic acid; |
| B-32 | 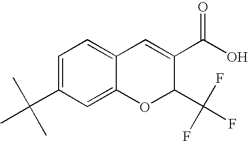 |
| | 7-(1,1-dimethylethyl)-2-trifluoromethyl-2H-1- |
| | benzopyran-3-carboxylic acid; |
| B-33 | 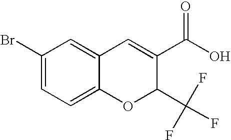 |
| | 6-bromo-2-trifluoromethyl-2H-1-benzopyran-3- |
| | carboxylic acid; |
| B-34 | 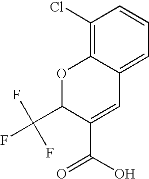 |
| | 8-chloro-2-trifluoromethyl-2H-1-benzopyran-3- |
| | carboxylic acid; |
| B-35 | 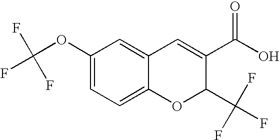 |
| | 6-trifluoromethoxy-2-trifluoromethyl-2H-1-benzopyran-3- |
| | carboxylic acid; |
| B-36 | 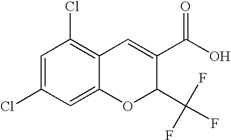 |
| | 5,7-dichloro-2-trifluoromethyl-2H-1-benzopyran-3- |
| | carboxylic acid; |
| B-37 | 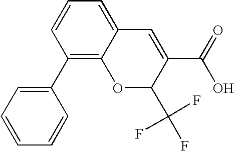 |
| | 8-phenyl-2-trifluoromethyl-2H-1-benzopyran-3- |
| | carboxylic acid; |
| B-38 | 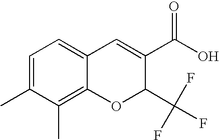 |
| | 7,8-dimethyl-2-trifluoromethyl-2H-1-benzopyran-3- |
| | carboxylic acid; |
| B-39 | 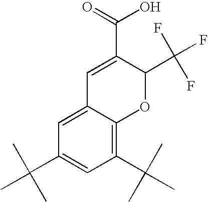 |
| | 6,8-bis(dimethylethyl)-2-trifluoromethyl-2H-1- |
| | benzopyran-3-carboxylic acid; |
| B-40 | 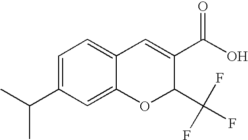 |
| | 7-(1-methylethyl)-2-trifluoromethyl-2H-1-benzopyran- |
| | 3-carboxylic acid; |
| B-41 | 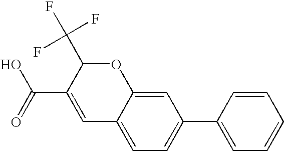 |
| | 7-phenyl-2-trifluoromethyl-2H-1-benzopyran-3- |
| | carboxylic acid; |
| B-42 | 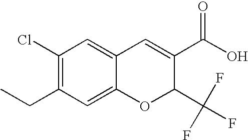 |
| | 6-chloro-7-ethyl-2-trifluoromethyl-2H-1-benzopyran-3- |
| | carboxylic acid; |
| B-43 | 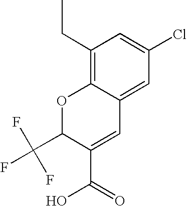 |
| | 6-chloro-8-ethyl-2-trifluoromethyl-2H-1-benzopyran-3- |
| | carboxylic acid; |
| B-44 | 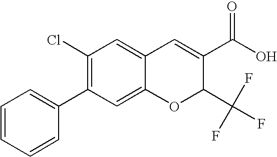 |
| | 6-chloro-7-phenyl-2-trifluoromethyl-2H-1-benzopyran-3- |
| | carboxylic acid; |
| B-45 | 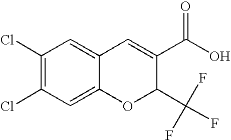 |
| | 6,7-dichloro-2-trifluoromethyl-2H-1-benzopyran-3- |
| | carboxylic acid; |
| B-46 | 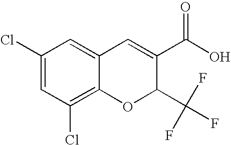 |
| | 6,8-dichloro-2-trifluoromethyl-2H-1-benzopyran-3- |
| | carboxylic acid; |
| B-47 | 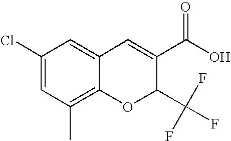 |
| | 6-chloro-8-methyl-2-trifluoromethyl-2H-1-benzopyran-3- |
| | carboxylic acid; |
| B-48 | 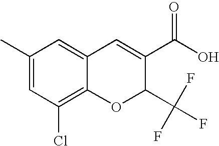 |
| | 8-chloro-6-methyl-2-trifluoromethyl-2H-1-benzopyran-3- |
| | carboxylic acid; |
| B-49 | 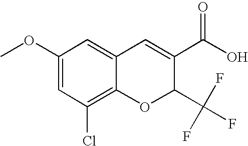 |
| | 8-chloro-6-methoxy-2-trifluoromethyl-2H-1-benzopyran-3- |
| | carboxylic acid; |
| B-50 | 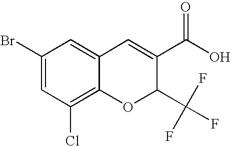 |
| | 6-bromo-8-chloro-2-trifluoromethyl-2H-1-benzopyran-3- |
| | carboxylic acid; |
| B-51 | 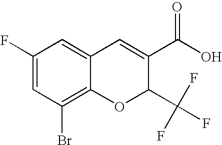 |
| | 8-bromo-6-fluoro-2-trifluoromethyl-2H-1-benzopyran-3- |
| | carboxylic acid; |
| B-52 | 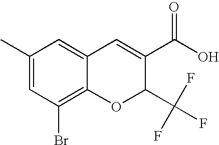 |
| | 8-bromo-6-methyl-2-trifluoromethyl-2H-1-benzopyran-3- |
| | carboxylic acid; |
| B-53 | 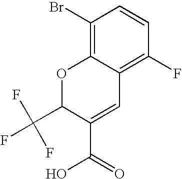 |
| | 8-bromo-5-fluoro-2-trifluoromethyl-2H-1-benzopyran-3- |
| | carboxylic acid; |
| B-54 | 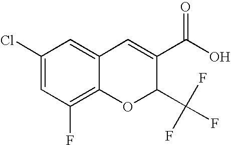 |
| | 6-chloro-8-fluoro-2-trifluoromethyl-2H-1-benzopyran-3- |
| | carboxylic acid; |
| B-55 | 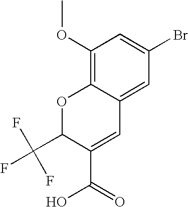 |
| | 6-bromo-8-methoxy-2-trifluoromethyl-2H-1-benzopyran-3- |
| | carboxylic acid; |
| B-56 | 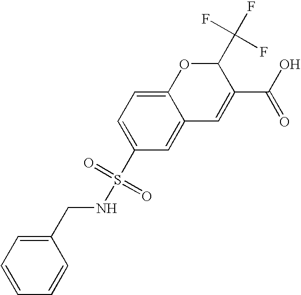 |
| | 6-[[(phenylmethyl)amino]sulfonyl]-2-trifluoromethyl-2H-1- |
| | benzopyran-3-carboxylic acid; |
| B-57 | 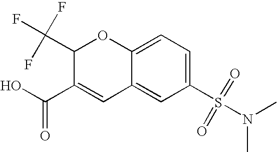 |
| | 6-[(dimethylamino)sulfonyl]-2-trifluoromethyl-2H-1- |
| | benzopyran-3-carboxylic acid; |
| B-58 | 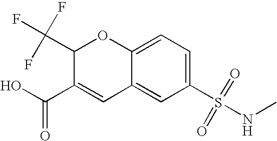 |
| | 6-[(methylamino)sulfonyl]-2-trifluoromethyl-2H-1- |
| | benzopyran-3-carboxylic acid; |
| B-59 | 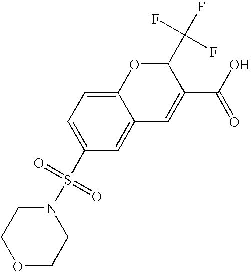 |
| | 6-[(4-morpholino)sulfonyl]-2-trifluoromethyl-2H-1- |
| | benzopyran-3-carboxylic acid; |
| B-60 | 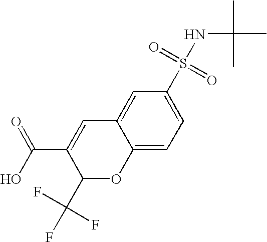 |
| | 6-[(1,1-dimethylethyl)aminosulfonyl]-2-trifluoromethyl- |
| | 2H-1-benzopyran-3-carboxylic acid; |
| B-61 | 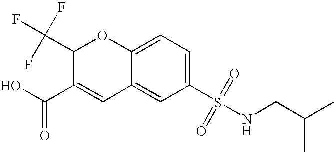 |
| | 6-[(2-methylpropyl)aminosulfonyl]-2-trifluoromethyl-2H- |
| | 1-benzopyran-3-carboxylic acid; |
| B-62 | 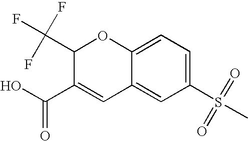 |
| | 6-methylsulfonyl-2-trifluoromethyl-2H-1-benzopyran-3- |
| | carboxylic acid; |
| B-63 | 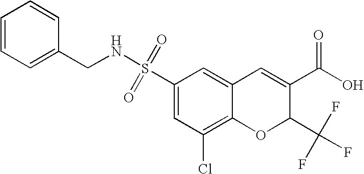 |
| | 8-chloro-6-[[(phenylmethyl)amino]sulfonyl]-2- |
| | trifluoromethyl-2H-1-benzopyran-3-carboxylic acid; |
| B-64 | 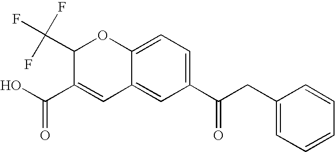 |
| | 6-phenylacetyl-2-trifluoromethyl-2H-1-benzopyran-3- |
| | carboxylic acid; |
| B-65 | 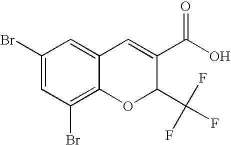 |
| | 6,8-dibromo-2-trifluoromethyl-2H-1-benzopyran-3- |
| | carboxylic acid; |
| B-66 | 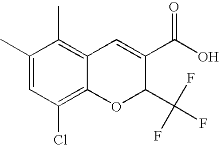 |
| | 8-chloro-5,6-dimethyl-2-trifluoromethyl-2H-1-benzopyran-3- |
| | carboxylic acid; |
| B-67 | 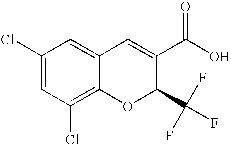 |
| | 6,8-dichloro-(S)-2-trifluoromethyl-2H-1-benzopyran-3- |
| | carboxylic acid; |
| B-68 | 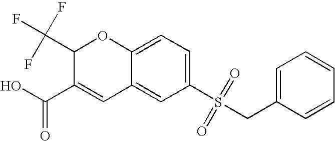 |
| | 6-benzylsulfonyl-2-trifluoromethyl-2H-1-benzopyran-3- |
| | carboxylic acid; |
| B-69 | 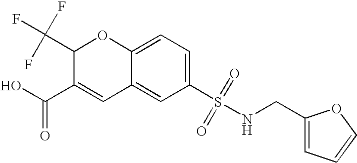 |
| | 6-[[N-(2-furylmethyl)amino]sulfonyl]-2-trifluoromethyl-2H-1- |
| | benzopyran-3-carboxylic acid; |
| B-70 | 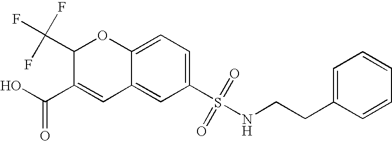 |
| | 6-[[N-(2-phenylethyl)amino]sulfonyl]-2-trifluoromethyl-2H-1- |
| | benzopyran-3-carboxylic acid; |
| B-71 | 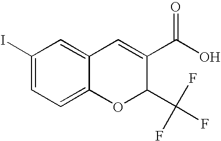 |
| | 6-iodo-2-trifluoromethyl-2H-1-benzopyran-3-carboxylic acid; |
| B-72 | 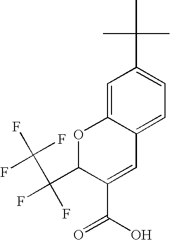 |
| | 7-(1,1-dimethylethyl)-2-pentafluoroethyl-2H-1-benzopyran-3- |
| | carboxylic acid; |
| B-73 | 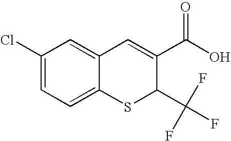 |
| | 6-chloro-2-trifluoromethyl-2H-1-benzothiopyran-3- |
| | carboxylic acid; |
| B-74 | 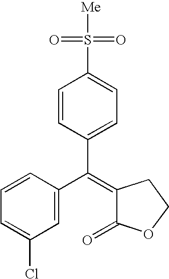 |
| | 3-[(3-Chloro-phenyl)-(4-methanesulfonyl-phenyl)-methylene]- |
| | dihydro-furan-2-one or BMS-347070; |
| B-75 | 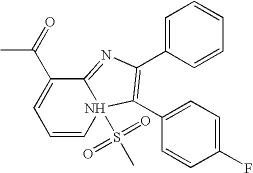 |
| | 8-acetyl-3-(4-fluorophenyl)-2-(4-methylsulfonyl)phenyl- |
| | imidazo(1,2-a)pyridine; |
| B-76 | 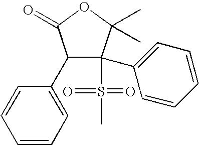 |
| | 5,5-dimethyl-4-(4-methylsulfonyl)phenyl-3-phenyl-2- |
| | (5H)-furanone; |
| B-77 | 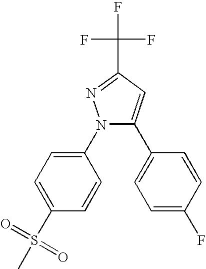 |
| | 5-(4-fluorophenyl)-1-[4-(methylsulfonyl)phenyl]- |
| | 3-(trifluoromethyl)pyrazole; |
| B-78 | 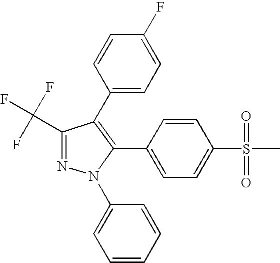 |
| | 4-(4-fluorophenyl)-5-[4-(methylsulfonyl)phenyl]-1- |
| | phenyl-3-(trifluoromethyl)pyrazole; |
| B-79 | 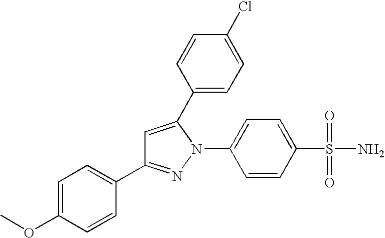 |
| | 4-(5-(4-chlorophenyl)-3-(4-methoxyphenyl)-1H-pyrazol-1- |
| | yl)benzenesulfonamide; |
| B-80 | 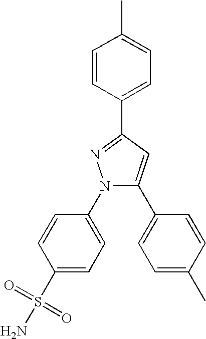 |
| | 4-(3,5-bis(4-methylphenyl)-1H-pyrazol-1- |
| | yl)benzenesulfonamide; |
| B-81 | 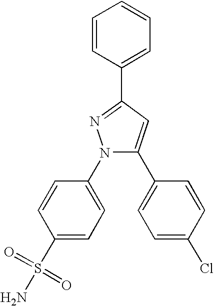 |
| | 4-(5-(4-chlorophenyl)-3-phenyl-1H-pyrazol-1- |
| | yl)benzenesulfonamide; |
| B-82 | 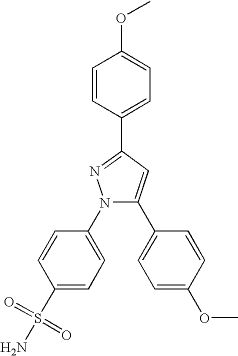 |
| | 4-(3,5-bis(4-methoxyphenyl)-1H-pyrazol-1- |
| | yl)benzenesulfonamide; |
| B-83 | 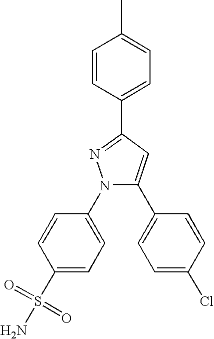 |
| | 4-(5-(4-chlorophenyl)-3-(4-methylphenyl)-1H- |
| | pyrazol-1-yl)benzenesulfonamide; |
| B-84 | 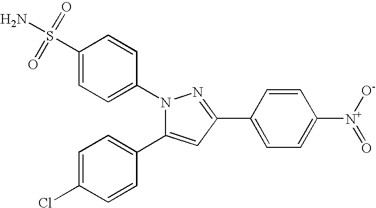 |
| | 4-(5-(4-chlorophenyl)-3-(4-nitrophenyl)-1H-pyrazol- |
| | 1-yl)benzenesulfonamide; |
| B-85 | 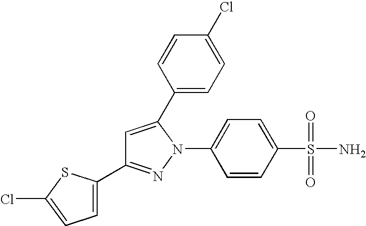 |
| | 4-(5-(4-chlorophenyl)-3-(5-chloro-2-thienyl)-1H- |
| | pyrazol-1-yl)benzenesulfonamide; |
| B-86 | 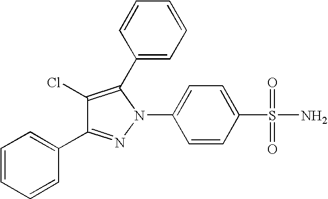 |
| | 4-(4-chloro-3,5-diphenyl-1H-pyrazol-1-yl)benzenesulfonamide; |
| B-87 | 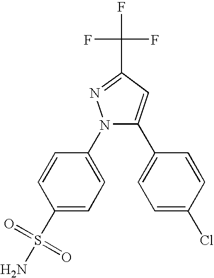 |
| | 4-[5-(4-chlorophenyl)-3-(trifluoromethyl)-1H- |
| | pyrazol-1-yl]benzenesulfonamide; |
| B-88 | 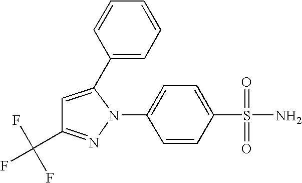 |
| | 4-[5-phenyl-3-(trifluoromethyl)-1H-pyrazol-1- |
| | yl]benzenesulfonamide; |
| B-89 | 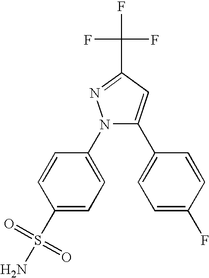 |
| | 4-[5-(4-fluorophenyl)-3-(trifluoromethyl)-1H- |
| | pyrazol-1-yl]benzenesulfonamide; |
| B-90 | 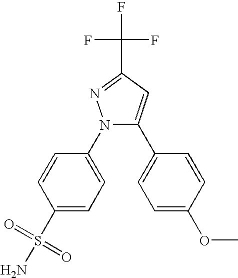 |
| | 4-[5-(4-methoxyphenyl)-3-(trifluoromethyl)-1H- |
| | pyrazol-1-yl]benzenesulfonamide; |
| B-91 | 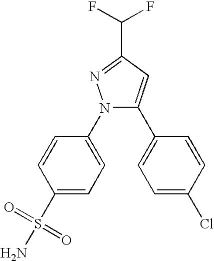 |
| | 4-[5-(4-chlorophenyl)-3-(difluoromethyl)-1H- |
| | pyrazol-1-yl]benzenesulfonamide; |
| B-92 | 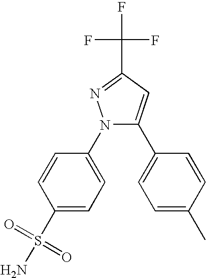 |
| | 4-[5-(4-methylphenyl)-3-(trifluoromethyl)-1H- |
| | pyrazol-1-yl]benzenesulfonamide; |
| B-93 | 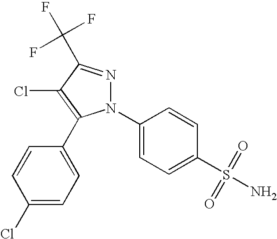 |
| | 4-[4-chloro-5-(4-chlorophenyl)-3-(trifluoromethyl)- |
| | 1H-pyrazol-1-yl]benzenesulfonamide; |
| B-94 | 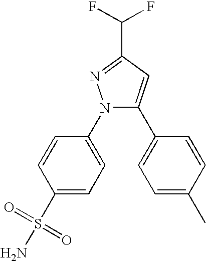 |
| | 4-[3-(difluoromethyl)-5-(4-methylphenyl)-1H-pyrazol-1- |
| | yl]benzenesulfonamide; |
| B-95 | 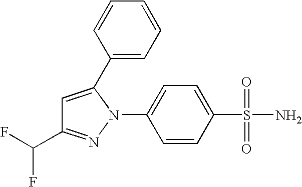 |
| | 4-[3-(difluoromethyl)-5-phenyl-1H-pyrazol-1- |
| | yl]benzenesulfonamide; |
| B-96 | 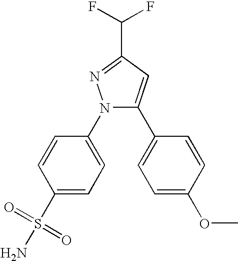 |
| | 4-[3-(difluoromethyl)-5-(4-methoxyphenyl)-1H- |
| | pyrazol-1-yl]benzenesulfonamide; |
| B-97 | 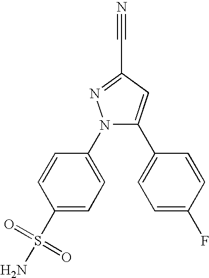 |
| | 4-[3-cyano-5-(4-fluorophenyl)-1H-pyrazol-1- |
| | yl]benzenesulfonamide; |
| B-98 | 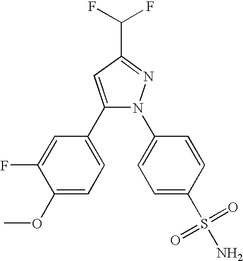 |
| | 4-[3-(difluoromethyl)-5-(3-fluoro-4-methoxyphenyl)- |
| | 1H-pyrazol-1-yl]benzenesulfonamide; |
| B-99 | 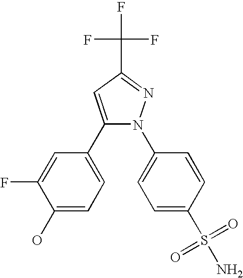 |
| | 4-[5-(3-fluoro-4-methoxyphenyl)-3-(trifluoromethyl)- |
| | 1H-pyrazol-1-yl]benzenesulfonamide; |
| B-100 | 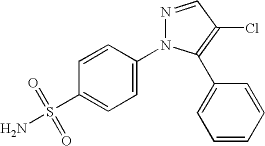 |
| | 4-[4-chloro-5-phenyl-1H-pyrazol-1-yl]benzenesulfonamide; |
| B-101 | 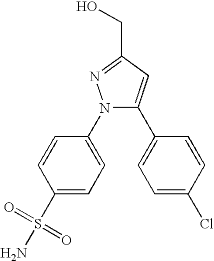 |
| | 4-[5-(4-chlorophenyl)-3-(hydroxymethyl)-1H-pyrazol- |
| | 1-yl]benzenesulfonamide; |
| B-102 | 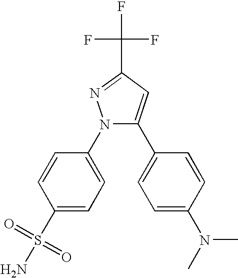 |
| | 4-[5-(4-(N,N-dimethylamino)phenyl)-3-(trifluoromethyl)- |
| | 1H-pyrazol-1-yl]benzenesulfonamide; |
| B-103 | 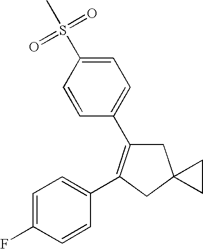 |
| | 5-(4-fluorophenyl)-6-[4-(methylsulfonyl)phenyl] |
| | Spiro[2.4]hept-5-ene; |
| B-104 | 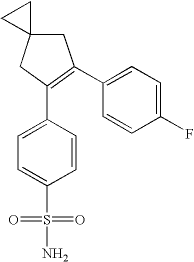 |
| | 4-[6-(4-fluorophenyl)spiro[2.4]hept-5-en-5- |
| | yl]benzenesulfonamide; |
| B-105 | 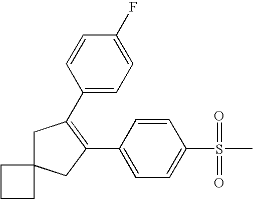 |
| | 6-(4-fluorophenyl)-7-[4-(methylsulfonyl)phenyl] |
| | spiro[3.4]oct-6-ene; |
| B-106 | 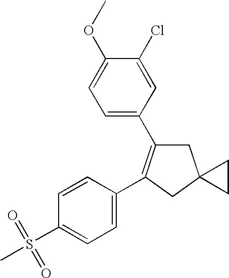 |
| | 5-(3-chloro-4-methoxyphenyl)-6-[4-(methylsulfonyl) |
| | phenyl]spiro[2.4]hept-5-ene; |
| B-107 | 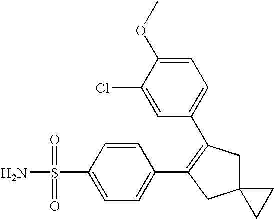 |
| | 4-[6-(3-chloro-4-methoxyphenyl)spiro[2.4]hept- |
| | 5-en-5-yl]benzenesulfonamide; |
| B-108 | 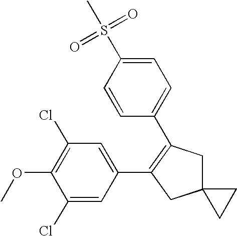 |
| | 5-(3,5-dichloro-4-methoxyphenyl)-6-[4- |
| | (methylsulfonyl)phenyl]Spiro[2.4]hept-5-ene; |
| B-109 | 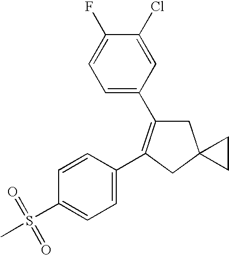 |
| | 5-(3-chloro-4-fluorophenyl)-6-[4- |
| | (methylsulfonyl)phenyl]spiro[2.4]hept-5-ene; |
| B-110 | 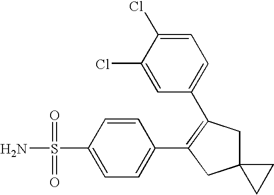 |
| | 4-[6-(3,4-dichlorophenyl)spiro[2.4]hept-5- |
| | en-5-yl]benzenesulfonamide; |
| B-111 | 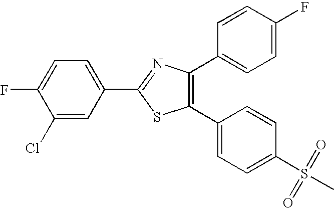 |
| | 2-(3-chloro-4-fluorophenyl)-4-(4-fluorophenyl)-5- |
| | (4-methylsulfonylphenyl)thiazole; |
| B-112 | 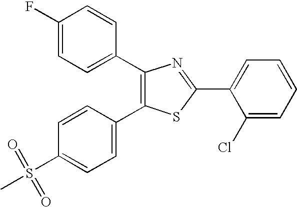 |
| | 2-(2-chlorophenyl)-4-(4-fluorophenyl)-5-(4- |
| | methylsulfonylphenyl)thiazole; |
| B-113 | 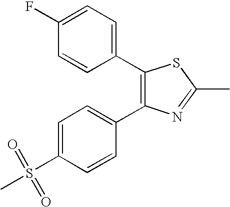 |
| | 5-(4-fluorophenyl)-4-(4-methylsulfonylphenyl)-2- |
| | methylthiazole; |
| B-114 | 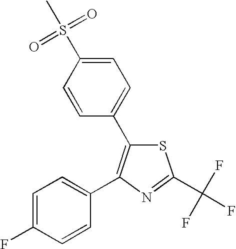 |
| | 4-(4-fluorophenyl)-5-(4-methylsulfonylphenyl)-2- |
| | trifluoromethylthiazole; |
| B-115 | 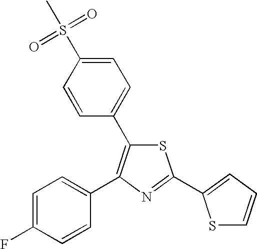 |
| | 4-(4-fluorophenyl)-5-(4-methylsulfonylphenyl)-2-(2- |
| | thienyl)thiazole; |
| B-116 | 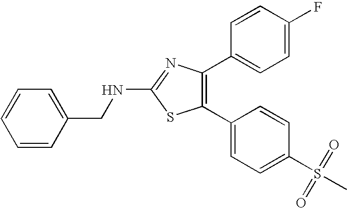 |
| | 4-(4-fluorophenyl)-5-(4-methylsulfonylphenyl)-2- |
| | benzylaminothiazole; |
| B-117 | 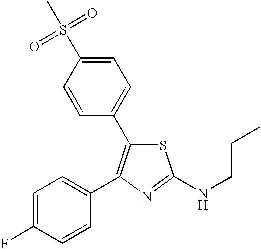 |
| | 4-(4-fluorophenyl)-5-(4-methylsulfonylphenyl)-2- |
| | (1-propylamino)thiazole; |
| B-118 | 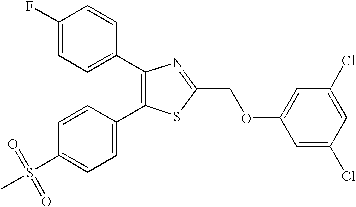 |
| | 2-((3,5-dichlorophenoxy)methyl)-4-(4-fluorophenyl)- |
| | 5-[4-(methylsulfonyl)phenyl]thiazole; |
| B-119 | 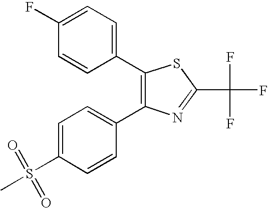 |
| | 5-(4-fluorophenyl)-4-(4-methylsulfonylphenyl)-2- |
| | trifluoromethylthiazole; |
| B-120 | 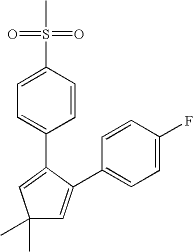 |
| | 1-methylsulfonyl-4-[1,1-dimethyl-4-(4- |
| | fluorophenyl)cyclopenta-2,4-dien-3-yl]benzene; |
| B-121 | 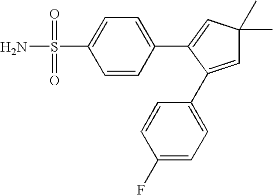 |
| | 4-[4-(4-fluorophenyl)-1,1-dimethylcyclopenta-2,4- |
| | dien-3-yl]benzenesulfonamide; |
| B-122 | 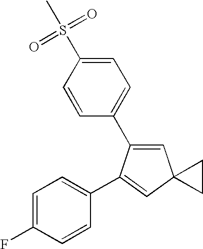 |
| | 5-(4-fluorophenyl)-6-[4-(methylsulfonyl)phenyl] |
| | spiro[2.4]hepta-4,6-diene; |
| B-123 | 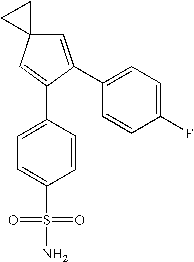 |
| | 4-[6-(4-fluorophenyl)spiro[2.4]hepta-4,6-dien- |
| | 5-yl]benzenesulfonamide; |
| B-124 | 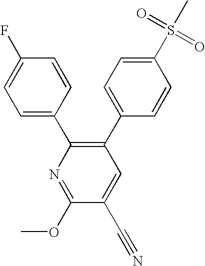 |
| | 6-(4-fluorophenyl)-2-methoxy-5-[4-(methylsulfonyl) |
| | phenyl]-pyridine-3-carbonitrile; |
| B-125 | 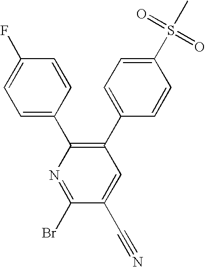 |
| | 2-bromo-6-(4-fluorophenyl)-5-[4-(methylsulfonyl) |
| | phenyl]-pyridine-3-carbonitrile; |
| B-126 | 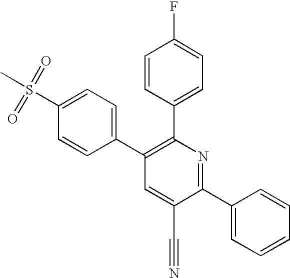 |
| | 6-(4-fluorophenyl)-5-[4-(methylsulfonyl)phenyl]- |
| | 2-phenyl-pyridine-3-carbonitrile; |
| B-127 | 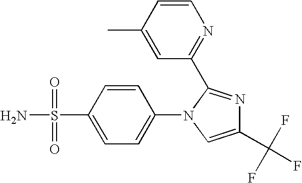 |
| | 4-[2-(4-methylpyridin-2-yl)-4-(trifluoromethyl)- |
| | 1H-imidazol-1-yl]benzenesulfonamide; |
| B-128 | 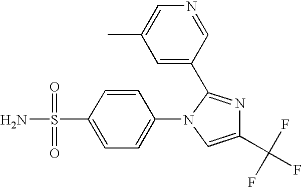 |
| | 4-[2-(5-methylpyridin-3-yl)-4-(trifluoromethyl)-1H- |
| | imidazol-1-yl]benzenesulfonamide; |
| B-129 | 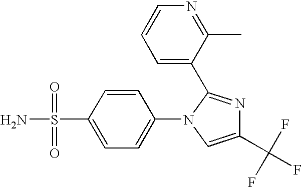 |
| | 4-[2-(2-methylpyridin-3-yl)-4-(trifluoromethyl)-1H- |
| | imidazol-1-yl]benzenesulfonamide; |
| B-130 | 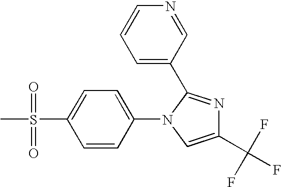 |
| | 3-[1-[4-(methylsulfonyl)phenyl]-4-(trifluoromethyl)- |
| | 1H-imidazol-2-yl]pyridine; |
| B-131 | 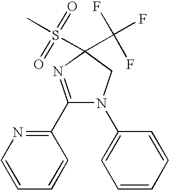 |
| | 2-[1-[4-(methylsulfonyl)phenyl-4-(trifluoromethyl)]- |
| | 1H-imidazol-2-yl]pyridine; |
| B-132 | 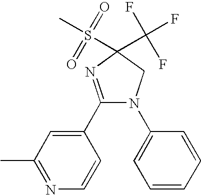 |
| | 2-methyl-4-[1-[4-(methylsulfonyl)phenyl-4-(trifluoromethyl)]- |
| | 1H-imidazol-2-yl]pyridine; |
| B-133 | 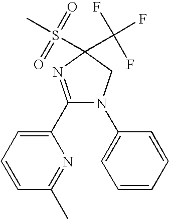 |
| | 2-methyl-6-[1-[4-(methylsulfonyl)phenyl-4-(trifluoromethyl)]- |
| | 1H-imidazol-2-yl]pyridine; |
| B-134 | 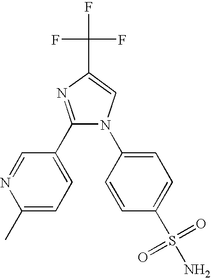 |
| | 4-[2-(6-methylpyridin-3-yl)-4-(trifluoromethyl)-1H- |
| | imidazol-1-yl]benzenesulfonamide; |
| B-135 | 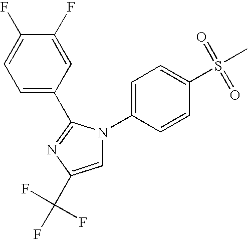 |
| | 2-(3,4-difluorophenyl)-1-[4-(methylsulfonyl)phenyl]-4- |
| | (trifluoromethyl)-1H-imidazole; |
| B-136 | 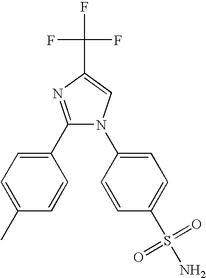 |
| | 4-[2-(4-methylphenyl)-4-(trifluoromethyl)-1H-imidazol- |
| | 1-yl]benzenesulfonamide; |
| B-137 | 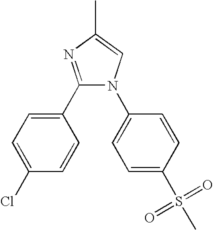 |
| | 2-(4-chlorophenyl)-1-[4-(methylsulfonyl)phenyl]-4- |
| | methyl-1H-imidazole; |
| B-138 | 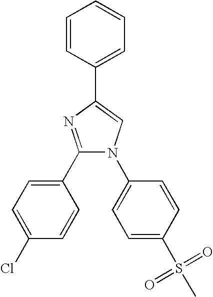 |
| | 2-(4-chlorophenyl)-1-[4-(methylsulfonyl)phenyl]-4- |
| | phenyl-1H-imidazole; |
| B-139 | 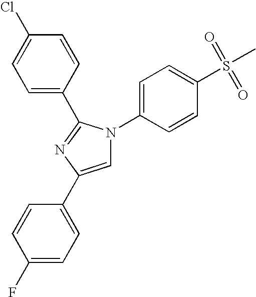 |
| | 2-(4-chlorophenyl)-4-(4-fluorophenyl)-1-[4- |
| | (methylsulfonyl)phenyl]-1H-imidazole; |
| B-140 | 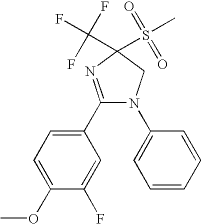 |
| | 2-(3-fluoro-4-methoxyphenyl)-1-[4- |
| | (methylsulfonyl)phenyl-4-(trifluoromethyl)]-1H-imidazole; |
| B-141 | 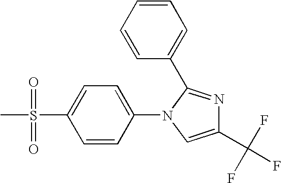 |
| | 1-[4-(methylsulfonyl)phenyl]-2-phenyl-4- |
| | trifluoromethyl-1H-imidazole; |
| B-142 | 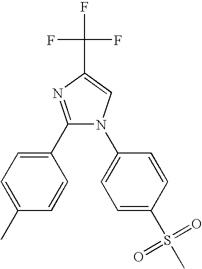 |
| | 2-(4-methylphenyl)-1-[4-(methylsulfonyl)phenyl]-4- |
| | trifluoromethyl-1H-imidazole; |
| B-143 | 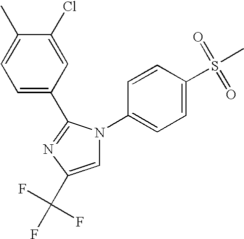 |
| | 4-[2-(3-chloro-4-methylphenyl)-4-(trifluoromethyl)-1H- |
| | imidazol-1-yl]benzenesulfonamide; |
| B-144 | 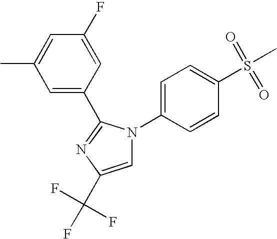 |
| | 2-(3-fluoro-5-methylphenyl)-1-[4-(methylsulfonyl)phenyl]- |
| | 4-(trifluoromethylyl)-1H-imidazole; |
| B-145 | 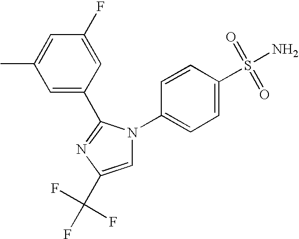 |
| | 4-[2-(3-fluoro-5-methylphenyl)-4-(trifluoromethyl)-1H- |
| | imidazol-1-yl]benzenesulfonamide; |
| B-146 | 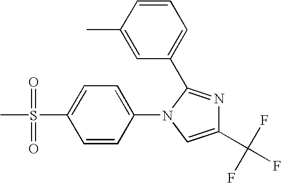 |
| | 2-(3-methylphenyl)-1-[4-(methylsulfonyl)phenyl]-4- |
| | trifluoromethyl-1H-imidazole; |
| B-147 | 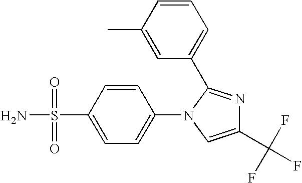 |
| | 4-[2-(3-methylphenyl)-4-trifluoromethyl-1H-imidazol-1- |
| | yl]benzenesulfonamide; |
| B-148 | 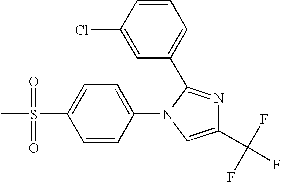 |
| | 1-[4-(methylsulfonyl)phenyl]-2-(3-chlorophenyl)-4- |
| | trifluoromethyl-1H-imidazole; |
| B-149 | 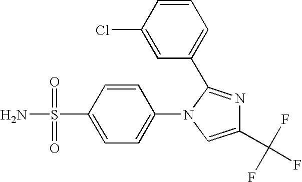 |
| | 4-[2-(3-chlorophenyl)-4-trifluoromethyl-1H-imidazol- |
| | 1-yl]benzenesulfonamide; |
| B-150 | 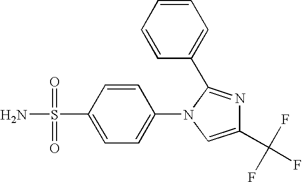 |
| | 4-[2-phenyl-4-trifluoromethyl-1H-imidazol-1- |
| | yl]benzenesulfonamide; |
| B-151 | 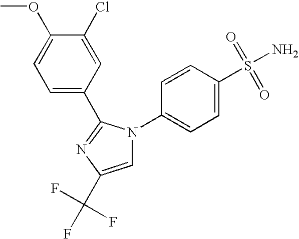 |
| | 4-[2-(4-methoxy-3-chlorophenyl)-4-trifluoromethyl-1H- |
| | imidazol-1-yl]benzenesulfonamide; |
| B-152 | 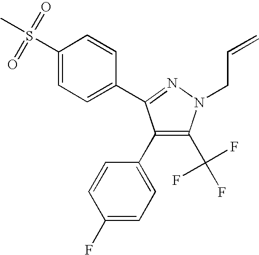 |
| | 1-allyl-4-(4-fluorophenyl)-3-[4-(methylsulfonyl) |
| | phenyl]-5-(trifluoromethyl)-1H-pyrazole; |
| B-153 | 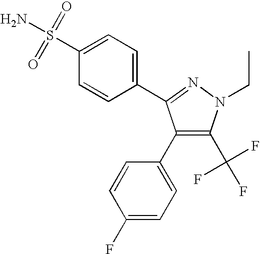 |
| | 4-[1-ethyl-4-(4-fluorophenyl)-5-(trifluoromethyl)-1H- |
| | pyrazol-3-yl]benzenesulfonamide; |
| B-154 | 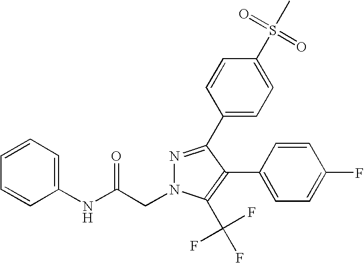 |
| | N-phenyl-[4-(4-fluorphenyl)-3-[4- |
| | (methylsulfonyl)phenyl]-5-(trifluoromethyl)-1H- |
| | pyrazol-1-yl]acetamide; |
| B-155 | 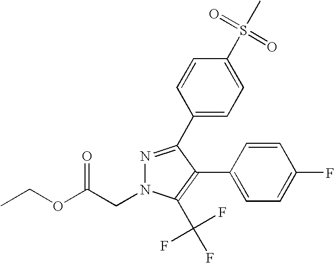 |
| | ethyl [4-(4-fluorophenyl)-3-[4-(methylsulfonyl)phenyl]- |
| | 5-(trifluoromethyl)-1H-pyrazol-1-yl]acetate; |
| B-156 | 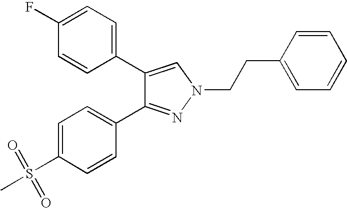 |
| | 4-(4-fluorophenyl)-3-[4-(methylsulfonyl)phenyl]-1- |
| | (2-phenylethyl)-1H-pyrazole; |
| B-157 | 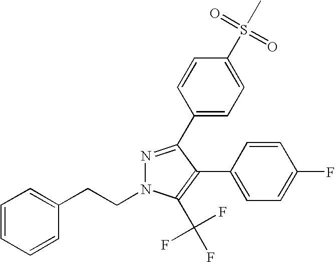 |
| | 4-(4-fluorophenyl)-3-[4-(methylsulfonyl)phenyl]-1-(2- |
| | phenylethyl)-5-(trifluoromethyl)pyrazole; |
| B-158 | 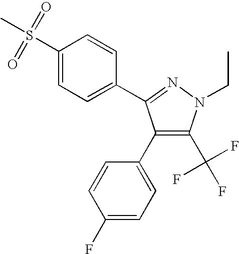 |
| | 1-ethyl-4-(4-fluorophenyl)-3-[4-(methylsulfonyl)phenyl]- |
| | 5-(trifluoromethyl)-1H-pyrazole; |
| B-159 | 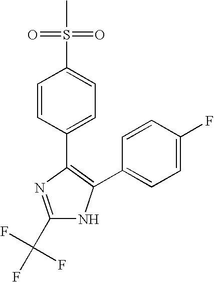 |
| | 5-(4-fluorophenyl)-4-(4-methylsulfonylphenyl)-2- |
| | trifluoromethyl-1H-imidazole; |
| B-160 | 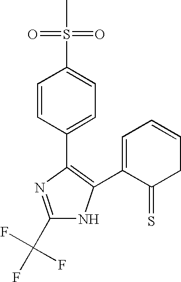 |
| | 4-[4-(methylsulfonyl)phenyl]-5-(2-thiophenyl)-2- |
| | (trifluoromethyl)-1H-imidazole; |
| B-161 | 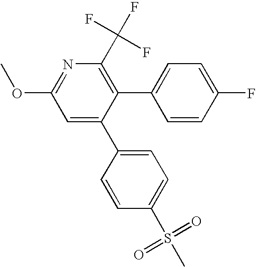 |
| | 5-(4-fluorophenyl)-2-methoxy-4-[4-(methylsulfonyl) |
| | phenyl]-6-(trifluoromethyl)pyridine; |
| B-162 | 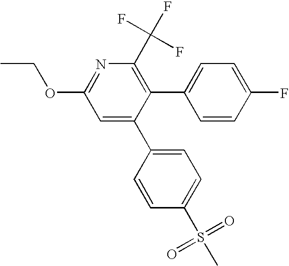 |
| | 2-ethoxy-5-(4-fluorophenyl)-4-[4-(methylsulfonyl) |
| | phenyl]-6-(trifluoromethyl)pyridine; |
| B-163 | 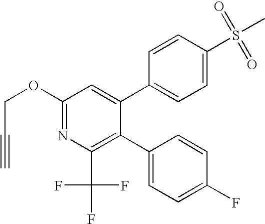 |
| | 5-(4-fluorophenyl)-4-[4-(methylsulfonyl)phenyl]-2- |
| | (2-propynyloxy)-6-(trifluoromethyl)pyridine; |
| B-164 | 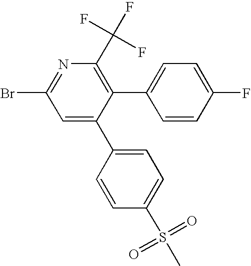 |
| | 2-bromo-5-(4-fluorophenyl)-4-[4-(methylsulfonyl)phenyl]- |
| | 6-(trifluoromethyl)pyridine; |
| B-165 | 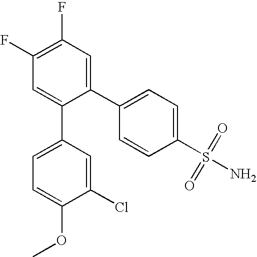 |
| | 4-[2-(3 -chloro-4-methoxyphenyl)-4,5-difluorophenyl] |
| | benzenesulfonamide; |
| B-166 | 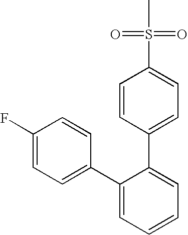 |
| | 1-(4-fluorophenyl)-2-[4-(methylsulfonyl)phenyl]benzene; |
| B-167 | 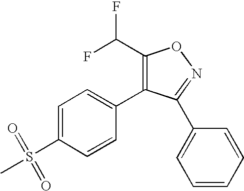 |
| | 5-difluoromethyl-4-(4-methylsulfonylphenyl)-3- |
| | phenylisoxazole; |
| B-168 | 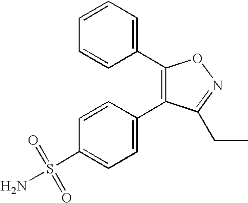 |
| | 4-[3-ethyl-5-phenylisoxazol-4-yl]benzenesulfonamide; |
| B-169 | 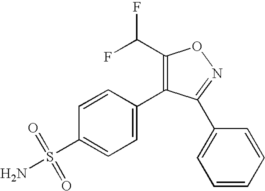 |
| | 4-[5-difluoromethyl-3-phenylisoxazol-4- |
| | yl]benzenesulfonamide; |
| B-170 | 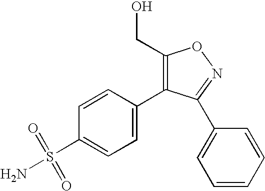 |
| | 4-[5-hydroxymethyl-3-phenylisoxazol-4- |
| | yl]benzenesulfonamide; |
| B-171 | 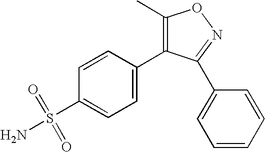 |
| | 4-[5-methyl-3-phenyl-isoxazol-4- |
| | yl]benzenesulfonamide; |
| B-172 | 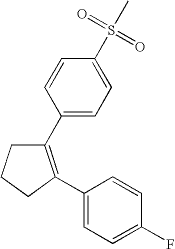 |
| | 1-[2-(4-fluorophenyl)cyclopenten-1-yl]-4- |
| | (methylsulfonyl)benzene; |
| B-173 | 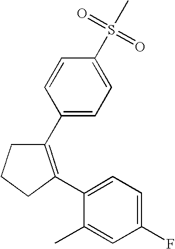 |
| | 1-[2-(4-fluoro-2-methylphenyl)cyclopenten-1- |
| | yl]-4-(methylsulfonyl)benzene; |
| B-174 | 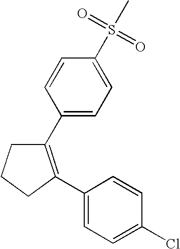 |
| | 1-[2-(4-chlorophenyl)cyclopenten-1-yl]-4- |
| | (methylsulfonyl)benzene; |
| B-175 | 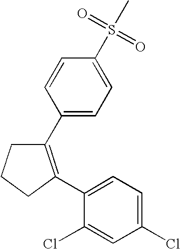 |
| | 1-[2-(2,4-dichlorophenyl)cyclopenten-1-yl]-4- |
| | (methylsulfonyl)benzene; |
| B-176 | 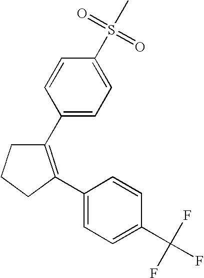 |
| | 1-[2-(4-trifluoromethylphenyl)cyclopenten-1- |
| | yl]-4-(methylsulfonyl)benzene; |
| B-177 | 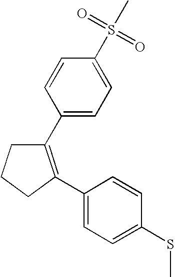 |
| | 1-[2-(4-methylthiophenyl)cyclopenten-1-yl]-4- |
| | (methylsulfonyl)benzene; |
| B-178 | 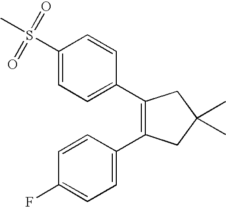 |
| | 1-[2-(4-fluorophenyl)-4,4-dimethylcyclopenten-1-yl]-4- |
| | (methylsulfonyl)benzene; |
| B-179 | 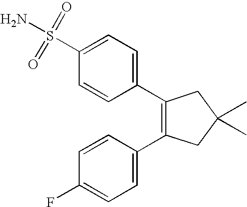 |
| | 4-[2-(4-fluorophenyl)-4,4-dimethylcyclopenten-1- |
| | yl]benzenesulfonamide; |
| B-180 | 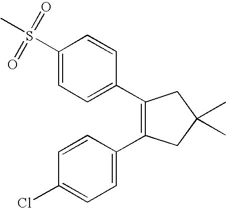 |
| | 1-[2-(4-chlorophenyl)-4,4-dimethylcyclopenten-1-yl]-4- |
| | (methylsulfonyl)benzene; |
| B-181 | 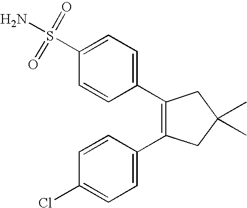 |
| | 4-[2-(4-chlorophenyl)-4,4-dimethylcyclopenten-1- |
| | yl]benzenesulfonamide; |
| B-182 | 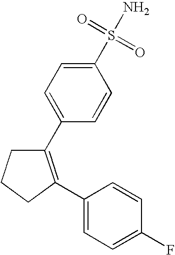 |
| | 4-[2-(4-fluorophenyl)cyclopenten-1- |
| | yl]benzenesulfonamide; |
| B-183 | 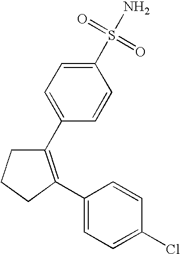 |
| | 4-[2-(4-chlorophenyl)cyclopenten-1- |
| | yl]benzenesulfonamide; |
| B-184 | 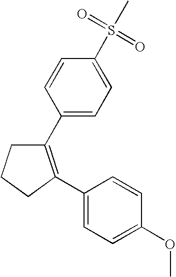 |
| | 1-[2-(4-methoxyphenyl)cyclopenten-1-yl]-4- |
| | (methylsulfonyl)benzene; |
| B-185 | 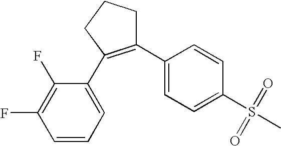 |
| | 1-[2-(2,3-difluorophenyl)cyclopenten-1-yl]-4- |
| | (methylsulfonyl)benzene; |
| B-186 | 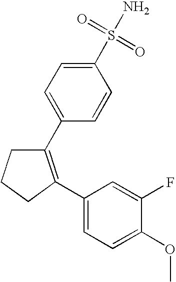 |
| | 4-[2-(3-fluoro-4-methoxyphenyl)cyclopenten-1- |
| | yl]benzenesulfonamide; |
| B-187 | 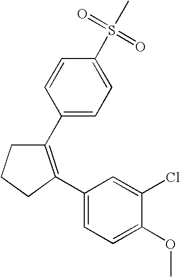 |
| | 1-[2-(3-chloro-4-methoxyphenyl)cyclopenten-1- |
| | yl]-4-(methylsulfonyl)benzene; |
| B-188 | 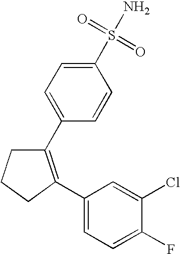 |
| | 4-[2-(3-chloro-4-fluorophenyl)cyclopenten-1- |
| | yl]benzenesulfonamide; |
| B-189 | 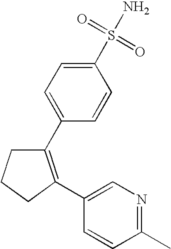 |
| | 4-[2-(2-methylpyridin-5-yl)cyclopenten-1-yl] |
| | benzenesulfonamide; |
| B-190 | 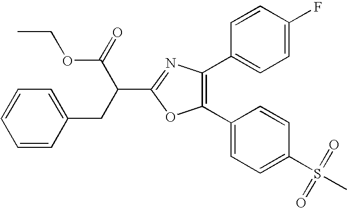 |
| | ethyl 2-[4-(4-fluorophenyl)-5-[4-(methylsulfonyl) |
| | phenyl]oxozol-2-yl]-2-benzyl-acetate; |
| B-191 | 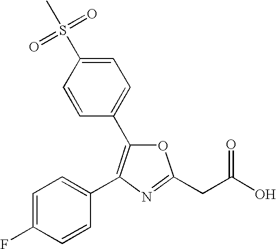 |
| | 2-[4-(4-fluorphenyl)-5-[4-(methylsulfonyl)phenyl] |
| | oxazol-2-yl]-2-benzyl-acetate; |
| B-192 | 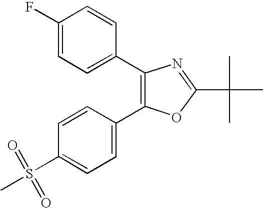 |
| | 2-(tert-butyl)-4-(4-fluorophenyl)-5-[4-(methylsulfonyl) |
| | phenyl]oxazole; |
| B-193 | 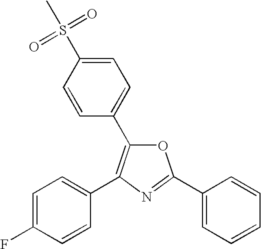 |
| | 4-(4-fluorophenyl)-5-[4-(methylsulfonyl)phenyl]-2- |
| | phenyloxazole; |
| B-194 | 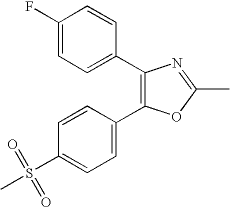 |
| | 4-(4-fluorophenyl)-2-methyl-5-[4- |
| | (methylsulfonl)phenyl]oxazole; |
| B-195 | 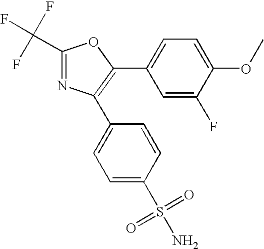 |
| | 4-[5-(3-fluoro-4-methoxyphenyl)-2-trifluoromethyl-4- |
| | oxazolyl]benzenesulfonamide; |
| B-196 | 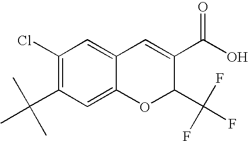 |
| | 6-chloro-7-(1,1-dimethylethyl)-2-trifluoromethyl-2H-1- |
| | benzopyran-3-carboxylic acid; |
| B-197 | 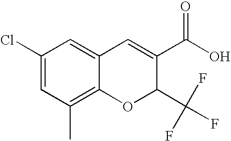 |
| | 6-chloro-8-methyl-2-trifluoromethyl-2H-1-benzopyran-3- |
| | carboxylic acid; |
| B-198 | 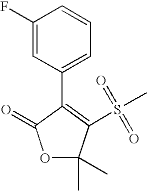 |
| | 5,5-dimethyl-3-(3-fluorophenyl)-4-methylsulfonyl- |
| | 2(5H)-furanone; |
| B-199 | 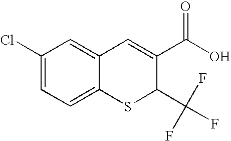 |
| | 6-chloro-2-trifluoromethyl-2H-1-benzothiopyran-3- |
| | carboxylic acid; |
| B-200 | 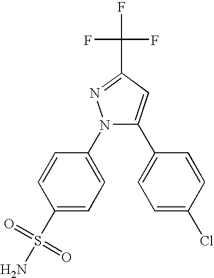 |
| | 4-[5-(4-chlorophenyl)-3-(trifluoromethyl)-1H- |
| | pyrazol-1-yl]benzenesulfonamide; |
| B-201 | 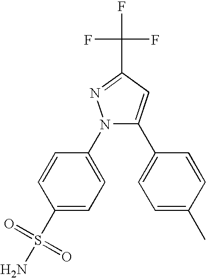 |
| | 4-[5-(4-methylphenyl)-3-(trifluoromethyl)-1H- |
| | pyrazol-1-yl]benzenesulfonamide; |
| B-202 | 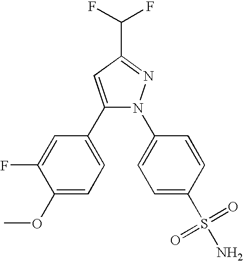 |
| | 4-[5-(3-fluoro-4-methoxyphenyl)-3-(difluoromethyl)- |
| | 1H-pyrazol-1-yl]benzenesulfonamide; |
| B-203 | 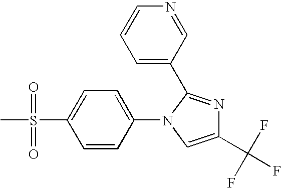 |
| | 3-[1-[4-(methylsulfonyl)phenyl]-4-trifluoromethyl- |
| | 1H-imidazol-2-yl]pyridine; |
| B-204 | 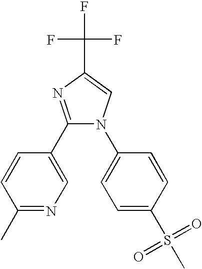 |
| | 2-methyl-5-[1-[4-(methylsulfonyl)phenyl]-4- |
| | trifluoromethyl-1H-imidazol-2-yl]pyridine; |
| B-205 | 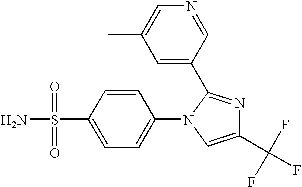 |
| | 4-[2-(5-methylpyridin-3-yl)-4-(trifluoromethyl)-1H- |
| | imidazol-1-yl]benzenesulfonamide; |
| B-206 | 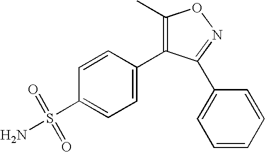 |
| | 4-[5-methyl-3-phenylisoxazo1-4-yl]benzenesulfonamide; |
| B-207 | 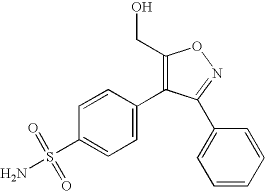 |
| | 4-[5-hydroxymethyl-3-phenylisoxazol-4- |
| | yl]benzenesulfonamide; |
| B-208 | 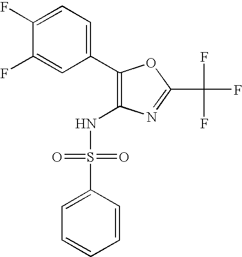 |
| | [2-trifluoromethyl-5-(3,4-difluorophenyl)-4- |
| | oxazolyl]benzenesulfonamide; |
| B-209 | 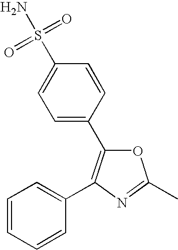 |
| | 4-[2-methyl-4-phenyl-5-oxazolyl]benzenesulfonamide; |
| B-210 | 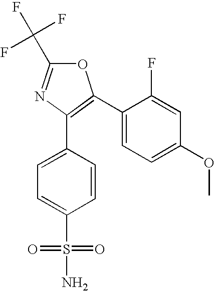 |
| | 4-[5-(2-fluoro-2-methoxyphenyl)-2-trifluoromethyl-4- |
| | oxazolyl]benzenesulfonamide; |
| B-211 | 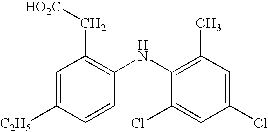 |
| | [2-(2,4-dichloro-6-methyl-phenylamino)-5-ethyl- |
| | phenyl]-acetic acid or COX 189 or Lumiracoxib |
| B-212 | 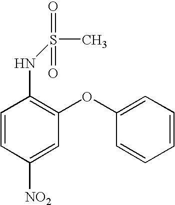 |
| | N-(4-Nitro-2-phenoxy-phenyl)-methanesulfonamide or |
| | Nimesulide |
| B-213 | 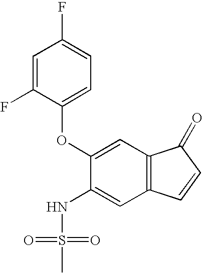 |
| | N-[6-(2,4-Difluoro-phenoxy)-1-oxo-inden-5-yl]- |
| | methanesulfonamide or Flosulide |
| B-214 | 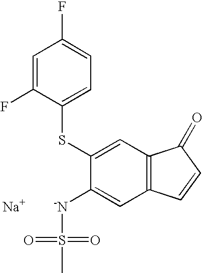 |
| | N-[6-(2,4-Difluoro-phenylsulfanyl)-1-oxo-1H-inden-5- |
| | yl]-methanesulfonamide, soldium salt, or L-745337 |
| B-215 | 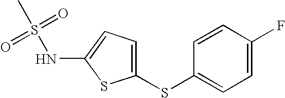 |
| | N-[5-(4-fluoro-phenylsulfanyl)-thiophen-2-yl]- |
| | methanesulfonamide or RWJ-63556 |
| B-216 | 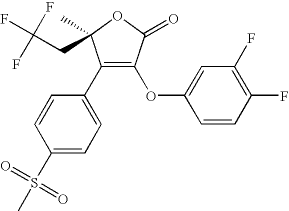 |
| | 3-(3,4-Difluoro-phenoxy)-4-(4-methanesulfonyl-Phenyl)-5- |
| | methyl-5-(2,2,2-trifluoro-ethyl)-5H-furan-2-one or L-784512 |
| B-217 | 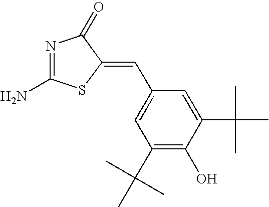 |
| | (5Z)-2-amino-5-[[3,5-bis(1,1-dimethylethyl)-4- |
| | hydroxyphenyl]methylene]-4(5H)-thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 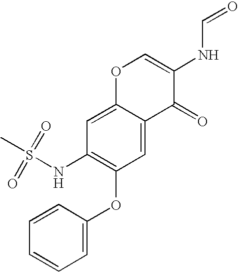 |
| | N-[3-(formylamino)-4-oxo-6-phenoxy-4H-1-benzopyran-7- |
| | yl]-methanesulfonamide or T614 |
| B-225 | D-1367 |
| B-226 | L-748731 |
| B-227 | 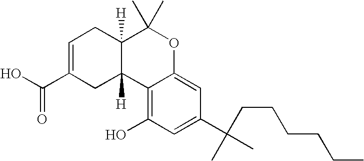 |
| | (6aR,10aR)-3-(1,1-dimethylheptyl)-6a,7,10,10a-tetrahydro-1- |
| | hydroxy-6,6-dimethyl-6H-dibenzo[b,d]pyran-9- |
| | carboxylic acid or CT 3 |
| | CT3 |
| B-228 | CGP-28238 |
| B-229 | 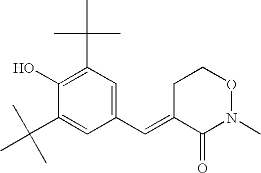 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| B-231 | 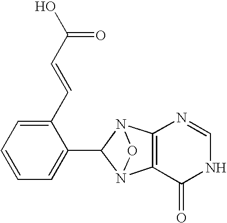 |
| | 2-(6-dioxo-9H-purin-8-yl)cinnamic acid |
| B-232 | S-2474 |

Drugs suitable for use as second drugs in combination with the COX 2 inhibitors of the present invention include, but are not limited to, neurotrophic factors, dopaminergics, dopamine agonists, neuronal nicotinic receptor agonists, k opioid receptor agonists, adenosine antagonists, glutamate antagonists, dopamine transport inhibitors, anticholinergics, enzyme inhibitors, atypical neuroletics, selective serotonin reuptake inhibitors, monoamine reuptake inhibitors, anti-apoptotics, adrenocortical steroids, antioxidants, drugs offsetting side effects of PD medications, and drugs that otherwise treat PD.

The various categories of second drugs disclosed above, without being limited to any particular theory of their physiological interactions, can be described generally according to the mechanisms by which they are currently believed to function. However, it is understood that the second drugs listed in Table 2 may function by additional means or in a different manner from that associated with the decriptions for their broader categories within which they are classified. The second drugs may also function according to mechanisms as yet unkown.

Neurotrophic factors are characterized as having an ability to promote the repair or replacement of neurons or otherwise restore lost neuron function. Dopaminergics generally function by introducing, replenishing, or promoting the release of, brain dopamine. Dopamine agonists are generally agonists for any of the dopamine receptors D1-D5, particularly receptors D1 and/or D2, where such dopamine agonists can delay or prevent complications associated with PD by mimicking dopamine's role in the brain and/or enhancing brain dopamine levels. Neuronal nicotinic receptor agonists are generally those drugs that enhance striatal dopamine, the release of norepinephrine in the cortex, or the release of acetylcholine in the frontal cortex and hippocampus, possibly resulting in decreased motor and cognitive disturbances and/or beneficial neurotrophic effects. The K opioid receptor agonists are generally drugs that cause a presynaptic reduction in glutamate release, mitigating any of a number of PD symptoms, particularly in combination with levodopa. Adenosine antagonists generally suppress adenosine levels and may reduce output of striatal neurons projecting to the external segment of the globus pallidus (indirect pathway) by increased GABA-mediated feedback inhibition and reduced acetylcholine release. Glutamate antagonists generally suppress glutamate levels and may potentiate the effects of levodopa, reduce dyskinesias, and possibly offer neurotrophic effects. Dopamine transport inhibitors generally act to increase endogenous dopamine in the synaptic cleft and may reduce any of a number of symptoms of PD and possibly offer neuroprotective effects due to the blocking of the uptake of toxins into dopaminergic neurons. Anticholinergics may reduce the amount of acetylcholine in the corpus striatum of the brain to offset the lack of dopamine in the substantia nigra, or may function as acetycholine receptor antagonists. Enzyme inhibitors are generally inhibitors of monoamine oxidase A (MAO-A), monoamine oxidase B (MAO-B), or catechol-o-methyltransferase (COMT) or other enzymes that function in the metabolism of dopamine or otherwise are involved in dopamine synthesis or breakdown and which enzyme's inhibition ultimately leads to an enhanced amount of brain dopamine. Atypical neuroleptics are generally those drugs that reduce any of the neurological effects associated with PD, including bradykinesia, rigidity, tremor, restlessness (akathisia), etc. Selective serotonin reuptake inhibitors are a category of antidepressants that have been found to have therapeutic potential in treating the depression, dementia, and akinesia found in PD patients. Monoamine reuptake inhibitors intensify brain monoamine neurotransmitters (e.g., norepinephrine) at its post-synaptic receptor sites. Anti-apoptotics inhibit apoptosis and/or mitochondrial dysfunction, which are thought to be involved in the aetiology of neurodegenerative diseases. Adrenocortical steroids generally interact with specific receptor proteins to regulate the expression of corticosteriod-responsive genes, thereby changing the levels and array of proteins synthesized. Antioxidants may reduce the physiological tendency for the occurance of potentially cell-damaging oxidation reactions. Antioxidants include enzymes involved in the metabolism of oxygen radicals (e.g. O²⁻) or the inhibitors of ezymes leading to the production of such radicals. Antioxidants also include free radical scavengers that may react directly with oxygen radicals and chelating agents that bind trace metals that can catalyze oxidation reactions. Other drugs may be used primarily to offset the side effects of PD medications, which side effects may include nausea, vomiting, hallucinations, nervousness, diarrhea, and the like. Other drugs for the treatment of PD and/or its symptoms function by as yet unknown or undefined mechanisms. According to one embodiment of the present invention, such drugs (see Table 2) are administered in combination with the COX 2 inhibitors of Tables 1 and 1A. Also see Tables 2A-2B. Examples of drugs within the categories described above include, but are not limited to, those given in Table 2 below:

TABLE 2

| Category | Example | Name |
|---|---|---|
| Neurotrophic Factors | | |
| | C-1 | GPI-1046 |
| | C-2 | GDNF |
| Dopaminergics | | |
| | C-3 | levodopa |
| | C-4 | levodopa/carbidopa (Sinemet ® or |
| | | Madopar ®) |
| | C-5 | levodopa/benserazide |
| | C-6 | amantadine (Symmetrel ®) |
| Dopamine Agonists | | |
| | C-7 | sumanirole |
| | C-8 | pergolide (Permax ®) |
| | C-9 | pramipexole or its hydrochloride salt |
| | | (Mirapex ®) |
| | C-10 | ropinirole or its hydrochloride salt |
| | | (Requip ®) |
| | C-11 | bromocriptine (Parlodel ®) |
| | C-12 | lisuride or 9,10 dihydrolisuride |
| | C-13 | apomorphine or N- |
| | | propylnoraporphine |
| | C-14 | N-propyl noraporphine |
| | C-15 | PHNO |
| | C-16 | N-0437 (racemate) and N-0923 |
| | | (purified negative enantiomer) |
| | C-17 | cabergoline |
| | C-18 | ciladopa |
| | C-19 | ABT-431 |
| | C-20 | lergotrile |
| Neuronal Nicotinic | | |
| Receptor Agonists | | |
| | C-21 | SIB1508Y |
| | C-22 | ABT418 |
| K Opioid Receptor | | |
| Agonists | | |
| | C-23 | eradoline |
| | C-24 | U-69,593 |
| Adenosine Antagonists | | |
| | C-25 | KW6002 |
| Glutamate Antagonists | | |
| | C-26 | remacemide |
| | C-27 | dextromethorphan |
| | C-28 | riluzole |
| Dopamine Transport | | |
| Inhibitors | | |
| | C-29 | NS-2214 |
| Anticholinergics | | |
| | C-30 | benzhexol |
| | C-31 | trihexyphenidyl or its hydrochloride |
| | | salt (Artane ®) |
| | C-32 | benzotropine or its hydrochloride salt |
| | | (Cogentin ®) |
| | C-33 | diphenhydramine hydrochloride |
| | | (Benadryl ®) |
| | C-34 | orphenadrine or its hydrochloride |
| | | salt (Disipal ®) |
| | C-35 | chlorphenoxamine or its |
| | | hydrochloride salt (Phenoxene ®) |
| | C-36 | amitriptyline |
| | C-37 | doxepin |
| | C-38 | imipramine |
| | C-39 | nortriptyline |
| | C-40 | biperiden or its hydrochloride salt |
| | | (Akineton ®) |
| | C-41 | ethopropazine |
| | C-42 | procyclidine or its hydrochloride salt |
| | | (Kemadrin ®) |
| | C-43 | cycrimine or its hydrochloride salt |
| | C-44 | ethopropzaine or its hydrochloride |
| | | salt (Parsidol ®) |
| Enzyme Inhibitors | | |
| | C-45 | selegiline or its hydrochloride salt |
| | | (Elderpryl ® or Deprenyl ®) |
| | C-46 | lazabemide |
| | C-47 | rasagiline |
| | C-48 | moclobemide |
| | C-49 | entacapone (Contan ®) |
| | C-50 | tolcapone (Tasmar ®) |
| | C-51 | nitecapone |
| | C-52 | Ro 40-7592 |
| Atypical Neuroleptics | | |
| | C-53 | clozapine |
| | C-54 | risperidone |
| | C-55 | olanzapine (Zyprexa ®) |
| | C-56 | quetiapine |
| Selective Serotonin | | |
| Reuptake Inhibitors | | |
| | C-57 | fluoxetine (Prozac ®) |
| | C-58 | paroxetine (Paxil ®) |
| | C-59 | serataline (Zoloft ®) |
| Monoamine Reuptake | | |
| Inhibitors | | |
| | C-60 | reboxetine |
| Anti-Apoptotics | | |
| | C-61 | CGP 3466 |
| | C-62 | CEP-1347/KT-7515 |
| Adrenocortical | | |
| Steroids | | |
| | C-63 | oxandrolone |
| | C-64 | creatine |
| | C-65 | erythropeotin |
| | C-66 | dehydroepiandrosterone (DHEA) |
| Antioxidants | | |
| | C-67 | superoxide dismutase |
| | C-68 | glutathione |
| | C-69 | glutathione peroxidase |
| | C-70 | catalase |
| | C-71 | nitric oxide synthase |
| | C-72 | tocopherol (Vitamin E) |
| | C-73 | ascorbic acid (Vitamin C) |
| | C-74 | selenium |
| | C-75 | acetylcysteine |
| | C-76 | seleginine (Deprenyl ®) |
| | C-77 | pycnogenol |
| | C-78 | co-enzyme Q10 |
| | C-79 | beta carotene |
| Drugs Offsetting Side | | |
| Effects of PD | | |
| Medications | | |
| | C-80 | odansetron (Zofran ®) |
| Drugs That Otherwise | | |
| Treat PD | | |
| | C-81 | propanolol |
| | C-82 | memantadine |

Combinations of COX 2 inhibitor(s) and second drugs that may be administered, in accordance with the method of the present invention, for the treatment of PD are represented in the Tables 2A and 2B below:

TABLE 2A

| COX 2 | |
|---|---|
| Inhibitor | Second Drug(s) |
| I | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| II | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| III | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| IV | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| V | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-1 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-2 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-3 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-4 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-5 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-6 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-7 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-8 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-9 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-10 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-11 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-12 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-13 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-14 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-15 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-16 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-17 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-18 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-19 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-20 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-21 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-22 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-23 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-24 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-25 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-26 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-27 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-28 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-29 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-30 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-31 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-32 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-33 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-34 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-35 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-36 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-37 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-38 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-39 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-40 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-41 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-42 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-43 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-44 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-45 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-46 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-47 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-48 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-49 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-50 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-51 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-52 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-53 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-54 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-55 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-56 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-57 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-58 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-59 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-60 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-61 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-62 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-63 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-64 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-65 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-66 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-67 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-68 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-69 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-70 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-71 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-72 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-73 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-74 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-75 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-76 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-77 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-78 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-79 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-80 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-81 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-82 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-83 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-84 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-85 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-86 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-87 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-88 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-89 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-90 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-91 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-92 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-93 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-94 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-95 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-96 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-97 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-98 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-99 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-100 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-101 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-102 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-103 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-104 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-105 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-106 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-107 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-108 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-109 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-110 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-111 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-112 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-113 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-114 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-115 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-116 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-117 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-118 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-119 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-120 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-121 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-122 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-123 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-124 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-125 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-126 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-127 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-128 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-129 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-130 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-131 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-132 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-133 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-134 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-135 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-136 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-137 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-138 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-139 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-140 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-141 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-142 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-143 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-144 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-145 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-146 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-147 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-148 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-149 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-150 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-151 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-152 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-153 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-154 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-155 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-156 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-157 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-158 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-159 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-160 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-161 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-162 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-163 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-164 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-165 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-166 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-167 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-168 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-169 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-170 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-171 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-172 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-173 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-174 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-175 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-176 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-177 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-178 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-179 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-180 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-181 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-182 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-183 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-184 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-185 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-186 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-187 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-188 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-189 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-190 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-191 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-192 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-193 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-194 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-195 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-196 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-197 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-198 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-199 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-200 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-201 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-202 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-203 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-204 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-205 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-206 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-207 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-208 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-209 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-210 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-211 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-212 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-213 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-214 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-215 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-216 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-217 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-218 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-219 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-220 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-221 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-222 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-223 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-224 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-225 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-226 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-227 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-228 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-229 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-230 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-231 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |
| B-232 | In combination with one or more of C-1, C-2, C-3, C-4, C-5, |
| | C-6, C-7, C-8, C-9, C-10, C-11, C-12, C-13, C-14, C-15, C- |
| | 16, C-17, C-18, C-19, C-20, C-21, C-22, C-23, C-24, C-25, |
| | C-26, C-27, C-28, C-29, C-30, C-31, C-32, C-33, C-34, C-35, |
| | C-36, C-37, C-38, C-39, C-40, C-41, C-42, C-43, C-44, C-45, |
| | C-46, C-47, C-48, C-49, C-50, C-51, C-52, C-53, C-54, C-55, |
| | C-56, C-57, C-58, C-59, C-60, C-61, C-62, C-63, C-64, C-65, |
| | C-66, C-67, C-68, C-69, C-70, C-71, C-72, C-73, C-74, C-75, |
| | C-76, C-77, C-78, C-79, C-80, C-81 or C-82. |

TABLE 2B

| Second Drug | COX 2 Inhibitor |
|---|---|
| Neurotrophic Factors | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Dopaminergics | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Dopamine Agonists | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Neuronal Nicotinic | In combination with one or more of I, II, III, IV, |
| Receptor Agonists | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| K Opioid | In combination with one or more of I, II, III, IV, |
| Receptor Agonists | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Adenosine Antagonists | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Glutamate Antagonists | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Dopamine Transport | In combination with one or more of I, II, III, IV, |
| Inhibitors | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Anticholinergics | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Enzyme Inhibitors | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Atypical Neuroleptics | In combination with one or more of I, II, III, IV, |
| | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Selective Serotonin | In combination with one or more of I, II, III, IV, |
| Reuptake Inhibitors | V, B-1, B-2, B-3, B-4, B-5, B-6, B-7, B-8, B-9, |
| | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |
| Drugs Offsetting Side | In combination with one or more of I, II, III, IV, |
| Effects of PD | V, B-1, B-2, B-3, B-4, B-5, B-6, B-7, B-8, B-9, |
| Medications | B-10, B-11, B-12, B-13, B-14, B-15, B-16, B-17, |
| | B-18, B-19, B-20, B-21, B-22, B-23, B-24, B-25, |
| | B-26, B-27, B-28, B-29, B-30, B-31, B-32, B-33, |
| | B-34, B-35, B-36, B-37, B-38, B-39, B-40, B-41, |
| | B-42, B-43, B-44, B-45, B-46, B-47, B-48, B-49, |
| | B-50, B-51, B-52, B-53, B-54, B-55, B-56, B-57, |
| | B-58, B-59, B-60, B-61, B-62, B-63, B-64, B-65, |
| | B-66, B-67, B-68, B-69, B-70, B-71, B-72, B-73, |
| | B-74, B-75, B-76, B-77, B-78, B-79, B-80, B-81, |
| | B-82, B-83, B-84, B-85, B-86, B-87, B-88, B-89, |
| | B-90, B-91, B-92, B-93, B-94, B-95, B-96, B-97, |
| | B-98, B-99, B-100, B-101, B-102, B-103, B-104, |
| | B-105, B-106, B-107, B-108, B-109, B-110, |
| | B-111, B-112, B-113, B-114, B-115, B-116, |
| | B-117, B-118, B-119, B-120, B-121, B-122, |
| | B-123, B-124, B-125, B-126, B-127, B-128, |
| | B-129, B-130, B-131, B-132, B-133, B-134, |
| | B-135, B-136, B-137, B-138, B-139, B-140, |
| | B-141, B-142, B-143, B-144, B-145, B-146, |
| | B-147, B-148, B-149, B-150, B-151, B-152, |
| | B-153, B-154, B-155, B-156, B-157, B-158, |
| | B-159, B-160, B-161, B-162, B-163, B-164, |
| | B-165, B-166, B-167, B-168, B-169, B-170, |
| | B-171, B-172, B-173, B-174, B-175, B-176, |
| | B-177, B-178, B-179, B-180, B-181, B-182, |
| | B-183, B-184, B-185, B-186, B-187, B-188, |
| | B-189, B-190, B-191, B-192, B-193, B-194, |
| | B-195, B-196, B-197, B-198, B-199, B-200, |
| | B-201, B-202, B-203, B-204, B-205, B-206, |
| | B-207, B-208, B-209, B-210, B-211, B-212, |
| | B-213, B-214, B-215, B-216, B-217, B-218, |
| | B-219, B-220, B-221, B-222, B-223, B-224, |
| | B-225, B-226, B-227, B-228, B-229, B-230, |
| | B-231 or B-232. |

According to one embodiment, the invention is directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, wherein said COX 2 inhibitor comprises a chromene that is a substituted benzopyran, or is a chroman.

According to yet another embodiment, the invention is directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is selected from the group consisting of substituted benzothiopyrans, dihydroquinolines, or dihydronaphthalenes having the general Formula (I):

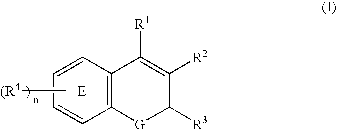

or an isomer, a pharmaceutically acceptable salt, ester, or prodrug thereof,

wherein n is an integer which is 0,1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with carbon atoms to which it is attached and the remainder of the ring E forms a naphthyl radical;

or an isomer, a pharmaceutically acceptable salt, ester, or prodrug thereof,

According to another embodiment, the invention is also directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the general Formula (II):

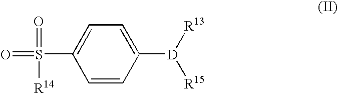

or an isomer, a pharmaceutically acceptable salt, ester, or prodrug thereof,

provided that Formula (II) is not celecoxib (B-18) or rofecoxib (B-21) as listed in Table 1A above,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

According to another embodiment, the invention is also directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the general Formula (II):

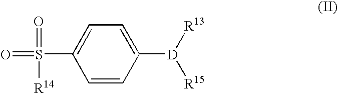

or an isomer, a pharmaceutically acceptable salt, ester, or prodrug thereof,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,

wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

According to another embodiment, the invention is also directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the general Formula (II):

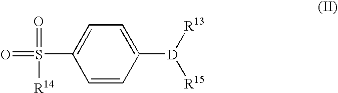

or an isomer, a pharmaceutically acceptable salt, ester, or prodrug thereof,

wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,

wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

According to another embodiment, the present invention is also directed to a novel method for the treatment of PD comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor comprises a phenylacetic acid derivative represented by the general Formula (III):

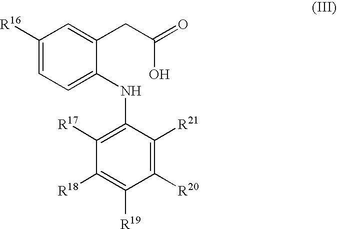

or an isomer, a pharmaceutically acceptable salt, ester, or prodrug thereof,

wherein:

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

According to another embodiment, the invention is directed to a method for the treatment of Parkinson's disease comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (IV):

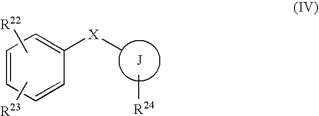

or an isomer, a pharmaceutically acceptable salt, ester, or prodrug thereof,

wherein:

X is O or S;

J is a carbocycle or a heterocycle;

R²² is NHSO₂CH₃or F;

R²³ is H, NO₂, or F; and

R²⁴ is H, NHSO₂CH₃, or (SO₂CH₃)C₆H₄.

According to another embodiment, the invention is directed to a method for the treatment of Parkinson's disease comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor has the structural Formula (V):

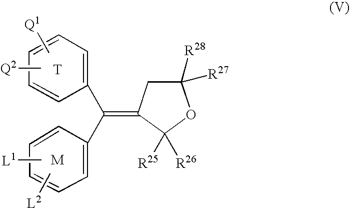

or an isomer, a pharmaceutically acceptable salt, ester, or prodrug thereof,

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

The present invention is also directed to a novel method of treating, improving or preventing a cyclooxygenase-2 mediated disorder in a subject, said method comprising treating the subject having or susceptible to said disorder with a therapeutically-effective amount of a pharmaceutical composition comprising any one of the cyclooxygenase-2-selective inhibitors described above and any one of the second drugs or categories of second drugs described above.

### DETAILED DESCRIPTION OF THE PREFERRED EMBODIMENTS

In accordance with the present invention, it has been discovered that PD can be treated by administering one or more cyclooxygenase-2 selective inhibitor(s) disclosed in Tables 1 and 1A above to subject(s) in need of such treatment, in combination with one or more second drugs disclosed in Table 2 or within the general categories of drugs disclosed in Table 2. The amount of the cyclooxygenase-2-selective inhibitor(s) and the second drug(s) used in the treatment of PD is selected so that the amount is therapeutically effective for the treatment, inhibition and/or prevention of PD.

The following definitions are provided in order to aid the reader in understanding the detailed description of the present invention.

The terms “hydrido” and “H” denote a single hydrogen atom. This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms.

Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The terms “heterocyclo”, “heterocyclyl”, and “heterocycle” embrace saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl and trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

As used herein, the term “carbocycle” means a hydrocarbon ring radical. Carbocyclic rings are monocyclic or are fused, bridged, or spiro polycyclic rings. Unless otherwise specified, monocyclic rings contain from 3 to about 9 atoms, preferably from about 4 to about 7 atoms, and most preferably 5 or 6 atoms. Polycyclic rings contain from about 7 to about 17 atoms, preferably from about 7 to about 14 atoms, and most preferably 9 or 10 atoms. Carbocyclic rings (carbocycles) may be substituted or unsubstituted.

As used herein, the term “purified” means partially purified and/or completely purified. Thus a “purified composition” may be either partially purified or completely purified. The COX 2 inhibitor(s), as well as the second drug(s), useful in the inventive method for treating PD, can be of any purity and quality such that the combinatiosn fo the COX 2 inhibitor(s) and second drug(s) is pharmaceutically acceptable.

In an embodiment of the present invention, any cyclooxygenase-2 selective inhibitor or isomer, pharmaceutically acceptable salt, ester, or prodrug thereof that meets the criteria described below can be used, along with any second drug, or isomer, pharmaceutically acceptable salt, ester, or prodrug thereof as described below, in the subject inventive method.

As used herein, the term “cyclooxygenase-2 inhibitor”, embraces compounds which selectively inhibit cyclooxygenase-2 over cyclooxygenase-1, and also includes pharmaceutically acceptable salts of those compounds.

In practice, the selectivity of a COX 2 inhibitor varies depending upon the condition under which the test is performed and on the inhibitors being tested. However, for the purposes of this specification, the selectivity of a COX 2 inhibitor can be measured as a ratio of the in vitro or in vivo IC₅₀ value for inhibition of COX 1, divided by the IC₅₀, value for inhibition of COX 2 (COX 1 IC₅₀/COX 2 IC₅₀). A COX 2 selective inhibitor is any inhibitor for which the ratio of COX 1 IC₅₀ to COX 2 IC₅₀ is greater than 1, preferably greater than 1.5, more preferably greater than 2, even more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity.

Preferred cyclooxygenase-2 selective inhibitors of the present invention have a cyclooxygenase-2 IC₅₀ of less than about 5 μM, more preferred of less than about 1 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of cyclooxygenase-2-selective inhibitors, as well as compounds that act as prodrugs for the second drug. As used herein in reference to COX 2 selective inhibitors and second drugs, the term “prodrug” refers to a chemical compound that is converted into an active COX 2 selective inhibitor by metabolic processes within the body. One example of a prodrug for a COX 2 selective inhibitor is parecoxib, which is a therapeutically effective prodrug of the tricyclic cyclooxygenase-2 selective inhibitor valdecoxib. An example of a preferred COX 2 selective inhibitor prodrug is sodium parecoxib.

The cyclooxygenase-2 selective inhibitor of the present invention can be, for example, the COX 2 selective inhibitor [2-(2,4-Dichloro-6-ethyl-3,5-dimethyl-phenylamino)-5-propyl-phenyl]-acetic acid, having Formula B-1, or an isomer or pharmaceutically acceptable salt, ester, or prodrug thereof.

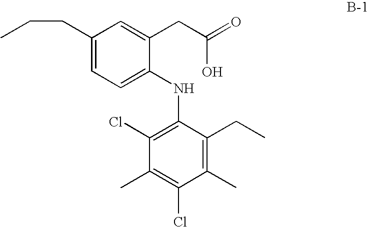

In another embodiment of the invention the cyclooxygenase-2 selective inhibitor can be the COX 2 selective inhibitor RS 57067 or 6-[[5-(4-chlorobenzoyl)-1,4-dimethyl-1H-pyrrol-2-yl]methyl]-3(2H)-pyridazinone, having Formula B-2 (CAS registry number 179382-91-3), or an isomer, a pharmaceutically acceptable salt, or prodrug thereof.

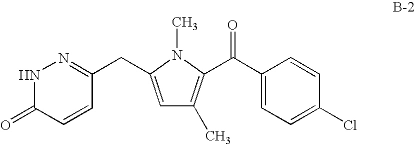

In a preferred embodiment of the invention the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having a structure shown by general Formulas (I)-(V), shown below, and possessing, by way of example and not limitation, the structures disclosed in Table 1, including the isomers, pharmaceutically acceptable salts, esters, and prodrugs thereof.

Furthermore, benzopyran COX 2 selective inhibitors useful in the practice of the present invention are described in U.S. Pat. Nos. 6,034,256 and 6,077,850.

Formula (I) is:

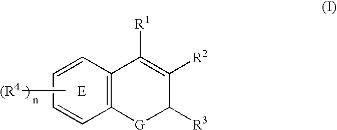

or an isomer, a pharmaceutically acceptable salt, ester, or prodrug thereof;

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof;

wherein:

n is an integer which is 0, 1, 2, 3 or 4;

wherein:

G is O, S or NR^{b};

R¹ is H;

R^{b} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

The cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

The cyclooxygenase-2 selective inhibitor used in connection with the method(s) of the present invention can also be a compound having the structure of Formula (I) or an isomer, a pharmaceutically acceptable salt, ester, or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6- membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

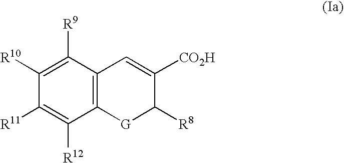

or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

The present invention is also directed to a novel method for the treatment of PD comprising administering to a subject in need thereof a therapeutically effective amount of a cyclooxygenase-2 selective inhibitor comprising BMS-347070 (B-74), ABT 963 (B-25), NS-398 (B-26), L-745337 (B-214), RWJ-63556 (B-215), or L-784512 (B-216).

Of the COX 2 inhibitors listed in Table 1A, those listed in Table 1B are chromene COX 2 inhibitors as indicated below:

TABLE 1B
Examples of Chromene COX 2 Selective Inhibitors

| | |
|---|---|
| No. | Structure (chromene COX 2 Inhibitor) |
| B-3 | 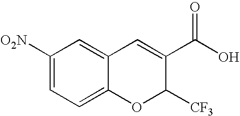 |
| | 6-Nitro-2-trifluoromethyl-2H-1- |
| | benzopyran-3-carboxylic acid |
| B-4 | 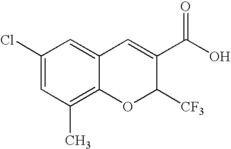 |
| | 6-Chloro-8-methyl-2-trifluoromethyl- |
| | 2H-1-benzopyran-3-carboxylic acid |
| B-5 | 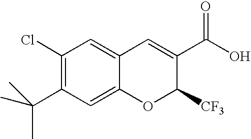 |
| | ((S)-6-Chloro-7-(1,1-dimethylethyl)-2-(trifluoro- |
| | methyl-2H-1-benzopyran-3-carboxylic acid |
| B-6 | 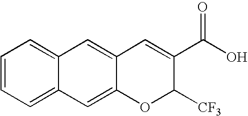 |
| | 2-Trifluoromethyl-2H-naphtho[2,3-b] |
| | pyran-3-carboxylic acid |
| B-7 | 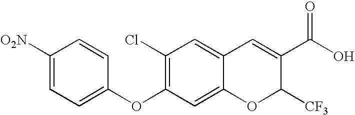 |
| | 6-Chloro-7-(4-nitrophenoxy)-2-(trifluoromethyl)-2H-1- |
| | benzopyran-3-carboxylic acid |
| B-8 | 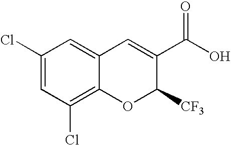 |
| | ((S)-6,8-Dichloro-2-(trifluoromethyl)- |
| | 2H-1-benzopyran-3-carboxylic acid |
| B-9 | 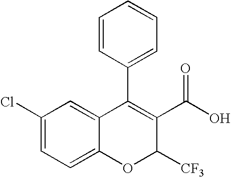 |
| | 6-Chloro-2-(trifluoromethyl)-4-phenyl-2H- |
| | 1-benzopyran-3-carboxylic acid |
| B-10 | 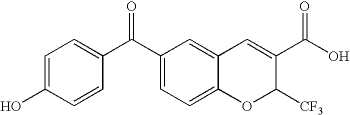 |
| | 6-(4-Hydroxybenzoyl)-2-(trifluoromethyl)- |
| | 2H-1-benzopyran-3-carboxylic acid |
| B-11 | 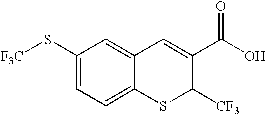 |
| | 2-(Trifluoromethyl)-6-[(trifluoromethyl)thio]- |
| | 2H-1-benzothiopyran-3-carboxylic acid |
| B-12 | 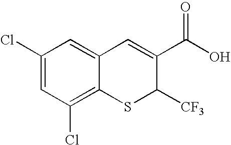 |
| | 6,8-Dichloro-2-trifluoromethyl-2H-1- |
| | benzothiopyran-3-carboxylic acid |
| B-13 | 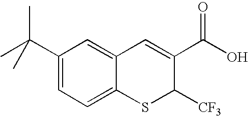 |
| | 6-(1,1-Dimethylethyl)-2-(trifluoromethyl)- |
| | 2H-1-benzothiopyran-3-carboxylic acid |
| B-14 | 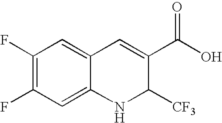 |
| | 6,7-Difluoro-1,2-dihydro-2-(trifluoro |
| | methyl)-3-quinolinecarboxylic acid |
| B-15 | 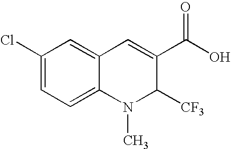 |
| | 6-Chloro-1,2-dihydro-1-methyl-2-(trifluoro |
| | methyl)-3-quinolinecarboxylic acid |
| B-16 | 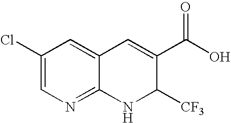 |
| | 6-Chloro-2-(trifluoromethyl)-1,2-dihydro |
| | [1,8]naphthyridine-3-carboxylic acid |
| B-17 | 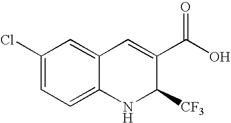 |
| | ((S)-6-Chloro-1,2-dihydro-2-(trifluoro |
| | methyl)-3-quinolinecarboxylic acid |

In a further preferred embodiment of the invention the cyclooxygenase inhibitor, when used in combination with any of the second drugs C-1 to C-82 or categories of second drugs (e.g., dopamine agonists) listed in Table 2, can be selected from the class of tricyclic cyclooxygenase-2 selective inhibitors represented by the general structure of Formula (II):

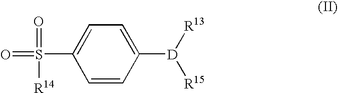

or an isomer, a pharmaceutically acceptable salt, ester, or prodrug thereof,

wherein:

D is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In a still more preferred embodiment of the invention, the tricyclic cyclooxygenase-2 selective inhibitor(s), for use in connection with the method(s) of the present invention and in combination with any of the second drugs C-1 to C-82 or categories of second drugs (e.g., dopamine agonists) listed in Table 2, are represented by the above Formula (II) and are selected from the group of compounds consisting of celecoxib (B-18), valdecoxib (B-19), deracoxib (B-20), rofecoxib (B-21), etoricoxib (MK-663; B-22), JTE-522 (B-23), or an isomer, a pharmaceutically acceptable salt, ester, or prodrug thereof.

TABLE 2
Examples of Tricyclic COX 2 Selective Inhibitors

| No. | Structure (Tricyclic COX 2 Inhibitors) |
|---|---|
| B-18 | 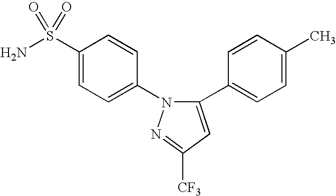 |
| | celecoxib |
| B-19 | 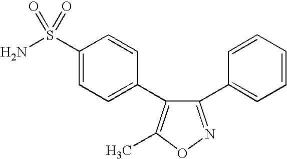 |
| | valdecoxib |
| B-20 | 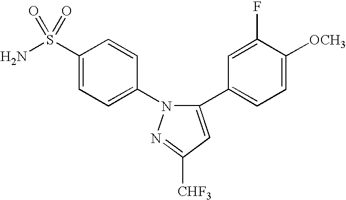 |
| | deracoxib |
| B-21 | 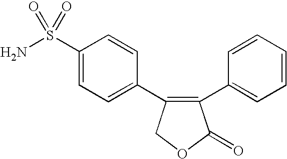 |
| | rofecoxib |
| B-22 | 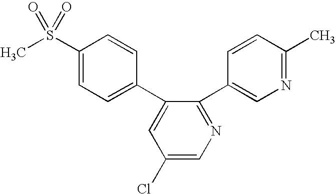 |
| | etoricoxib |
| B-23 | 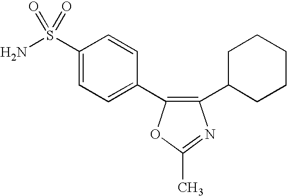 |
| | JTE-522 |

In an even more preferred embodiment of the invention, the COX 2 selective inhibitor, when used in combination with any of the second drugs C-1 to C-82 or categories of second drugs (e.g., dopamine agonists) listed in Table 2, is selected from the group consisting of celecoxib, rofecoxib and etoricoxib.

In another preferred embodiment of the invention, parecoxib, (B-24), which is a therapeutically effective prodrug of the tricyclic cyclooxygenase-2 selective inhibitor valdecoxib, (B-19), may be advantageously employed as a source of a cyclooxygenase inhibitor (See, e.g., U.S. Pat. No. 5,932,598) in connection with the method(s) in the present invention.

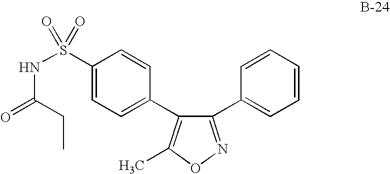

A preferred form of parecoxib is sodium parecoxib.

In another preferred embodiment of the invention, the compound ABT-963 having the formula (B-25) that has been previously described in International Publication number WO 00/24719, is another tricyclic cyclooxygenase-2 selective inhibitor which may be advantageously employed. in connection with the method(s) of the present invention.

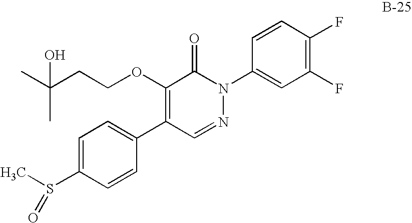

Another preferred cyclooxygenase-2 selective inhibitor that is useful in connection with the method(s) of the present invention is N-(2-cyclohexyloxynitrophenyl)-methane sulfonamide (NS-398)—having a structure shown below as B-26. Applications of this compound have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

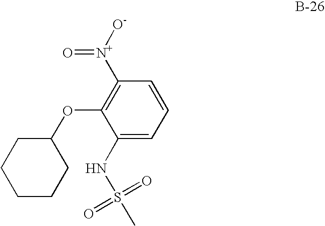

Other compounds that are useful for the cyclooxygenase-2 selective inhibitor in connection with the method(s) of the present invention include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2,4-dichloro-6-methyl-phenylamino)-5-ethyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231); or

S-2474 (B-232);

or an isomer, a pharmaceutically acceptable salt, ester or prodrug thereof, respectively.

Certain subgroups of the above-noted COX 2 inhibitors may be preferred for the treatment of PD which include, but are not limited to, B-1 to B-5, B-6 to B-10, B-11 to B-15, B-16 to B-20, B-21 to B-25, B-26 to B-30, B-31 to B-35, B-36-B-40, B-41 to B-45, B-46 to B-50, B-51 to B-55, B-56 to B-60, B-61 to B-65, B-66 to B-70, B-71 to B-75, B-76 to B-80, B-81 to B-85, B-B-86 to B-90, B-91 to B-95, B-96 to B-100, B-101 to B-105, B-106 to B-110, B-111 to B-115, B-116 to B-120, B-121 to B-125, B-126 to B-130, B-131 to B-135, B-136 to B-140, B-141 to B-145, B-146 to B-150, B-151 to B-155, B-156 to B-160, B-161 to B-165, B-166 to B-170, B-171 to B-175, B-176 to B-180, B-181 to B-185, B-186 to B-190, B-191 to B-195, B-196 to B-200, B-201 to B-205, B-206 to B-210, B-211 to B-215, B-216 to B-220, B-221 to B-225, B-226 to B-230, B-231-B-232 or combinations thereof.

In a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III):

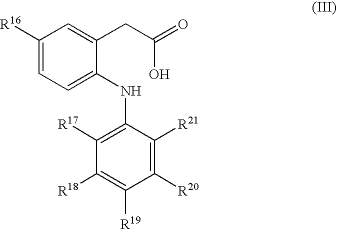

or an isomer, a pharmaceutically acceptable salt, ester, or prodrug thereof;

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R¹⁶ is ethyl;

R¹⁷ and R¹⁹ are chloro;

R 18 and R²⁰ are hydrogen; and

and R²¹ is methyl.

Drugs that may be effective as second drugs in combination with the COX 2 inhibitors include neurotrophic factors, dopaminergics, dopamine agonists, neuronal nicotinic receptor agonists, k opioid receptor agonists, adenosine antagonists, glutamate antagonists, dopamine transport inhibitors, anticholinergics, enzyme inhibitors, atypical neuroletics, selective serotonin reuptake inhibitors, monoamine reuptake inhibitors, anti-apoptotics, adrenocortical steroids, antioxidants, drugs offsetting side effects of PD medications, and drugs that otherwise treat PD by known or unknown mechanisms. Examples of neurotrophic factors include, but are not limited to, GPI-1046 and GDNF. Examples of dopaminergics include, but are not limited to, levodopa, levodopa/carbidopa (Sinemet® or Madopar®), levodopa/benserazide and amantadine (Symmetrel®). Examples of dopamine agonists include, but are not limited to, sumanirole, pergolide (Permax®), pramipexole or its hydrochloride salt (Mirapex®), ropinirole or its hydrochloride salt (Requip®), bromocriptine (Parlodel®), lisuride or 9,10 dihydrolisuride, apomorphine or N-propylnoraporphine, N-propyl noraporphine, PHNO, N-0437 (racemate) and N-0923 (purified negative enantiomer), cabergoline, ciladopa, ABT-431 and lergotrile. Examples of neuronal nicotinic receptor agonists include, but are not limited to, SIB1508Y and ABT418. Examples of k opioid receptor agonists include, but are not limited to, eradoline and U-69,593. Examples of adenosine antagonists include, but are not limited to, KW6002. Examples of glutamate antagonists include, but are not limited to, remacemide, dextromethorphan, and riluzole. Examples of dopamine transport inhibitors include, but are not limited to NS-2214. Examples of anticholinergics include, but are not limited to benzhexol, trihexyphenidyl or its hydrochloride salt (Artane®), benzotropine or its hydrochloride salt (Cogentin®), diphenhydramine hydrochloride (Benadryl®), orphenadrine or its hydrochloride salt (Disipal®), chlorphenoxamine or its hydrochloride salt (Phenoxene®), amitriptyline, doxepin, imipramine, nortriptyline, biperiden or its hydrochloride salt (Akineton®), ethopropazine, procyclidine or its hydrochloride salt (Kemadrin®), cycrimine or its hydrochloride salt, and ethopropzaine or its hydrochloride salt (Parsidol®). Examples of enzyme inhibitors include, but are not limited to, selegiline or its hydrochloride salt (Elderpryl® or Deprenyl®), lazabemide, rasagiline, moclobemide, entacapone (Contan®), tolcapone (Tasmar®), nitecapone and Ro 40-7592. Examples of atypical neuroletics include, but are not limted to clozapine, risperidone, olanzapine (Zyprexa®) and quetiapine. Examples of selective serotonin reuptake inhibitors include, but are not limited to, fluoxetine (Prozac®), paroxetine (Paxil®) and serataline (Zoloft®). A non-limiting example of a monoamine reuptake inhibitor is reboxetine. Reboxetine is 2-[α-(2-ethoxy) phenoxy-benzyl]morpholine and its preparation is described in U.S. Pat. No. 4,229,449. The structure of reboxetine is:

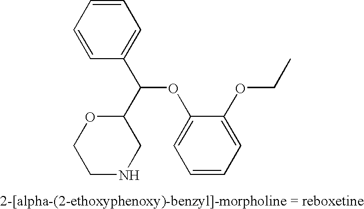

Reboxetine is described in one or more of the following U.S. Pat. Nos. as a reuptake inhibitor of the monoamine norepinephrine: 6,290,986 B 1; 6,229,010 B1; 6,096,742 B1; 6,191,133 B1; 6,184,222 B1; 6,117,855; 6,066,643; 6,028,070; 6,046,193; and 4,229,449. Examples of anti-apoptotics include, but are not limited to, CGP 3466 (described by Sagot et al., Br J Pharmacol October 2000; 131(4):721-8) and CEP-1347/KT-7515 (described by Saporito et al., JPET 288(2):421-7, 1999). Examples of adrenocortical steroids include, but are not limited to oxandrolone, creatine, erythropeotin and dehydroepiandrosterone (DHEA). Examples of antioxidants include, but are not limited to superoxide dismutase, glutathione, glutathione peroxidase, catalase, nitric oxide synthase, tocopherol (Vitamin E), ascorbic acid (Vitamin C), selenium, cetylcysteine, seleginine (Deprenyl®), pycnogenol, co-enzyme Q10 and beta carotene. Examples of drugs offsetting side effects of PD medications include, but are not limited to, odansetron (Zofran®). Examples of drugs that otherwise treat PD by known or unknown mechanisms include, but are not limited to, propanolol and memantadine.

The cyclooxygenase-2 selective inhibitors described previously may be referred to herein collectively as COX 2 selective inhibitors, or cyclooxygenase-2 selective inhibitors.

Cyclooxygenase-2 selective inhibitors as well as second drugs that are useful in the present invention can be supplied by any source as long as the combination of drugs is pharmaceutically acceptable. Cyclooxygenase-2-selective inhibitors and second drugs can be isolated and purified from natural sources or can be synthesized. The combination of the cyclooxygenase-2-selective inhibitor(s) and second drug(s) should be of a quality and purity that is conventional in the trade for use in pharmaceutical products.

In the present method, a subject in need of treatment of PD is treated with an amount of at least one COX 2 selective inhibitor and an amount of at least one second drug, where the amount of the COX 2 selective inhibitor together with the amount of second drug is sufficient to constitute a therapeutically effective amount for treating PD.

As used herein, an “effective amount” or “therapeutically effective amount” means the dose or effective amount to be administered to a patient and the frequency of administration to the subject which is sufficient to obtain a therapeutic effect as readily determined by one of ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically effective” indicates the capability of a combination of agents to prevent, or reduce the severity of, the disorder or its undesirable symptoms, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The amounts of the COX 2 selective inhibitor and the second drug that are used in the subject method may be amounts that, together, are sufficient to constitute an effective amount for PD treatment or prevention. In the present method, the amount of COX 2 selective inhibitor that is used in the novel method of treatment preferably ranges from about 0.001 to about 100 milligrams per day per kilogram of body weight of the subject (mg/day.kg), more preferably from about 0.05 to about 50 mg/day.kg, even more preferably from about 1 to about 20 mg/day.kg. The amount of the second drug, used in combination with the COX 2 selective inhibitor, preferably ranges from about 0.001 to about 100 milligrams per day per kilogram of body weight of the subject (mg/day.kg), more preferably from about 0.1 to about 10 mg/day.kg, even more preferably from about 0.5 to about 2 mg/day.kg. Preferably, the weight ratio of the amount(s) of COX 2 inhibitor(s) administered to the amount(s) of second drug(s) administered is from about 0.002 to about 10, more preferably from about 0.1 to about 5.

When the COX 2 selective inhibitor comprises rofecoxib, it is preferred that the amount used is within a range of from about 0.15 to about 1.0 mg/day.kg, and even more preferably from about 0.18 to about 0.4 mg/day.kg.

When the COX 2 selective inhibitor comprises etoricoxib, it is preferred that the amount used is within a range of from about 0.5 to about 5 mg/day.kg, and even more preferably from about 0.8 to about 4 mg/day.kg.

When the COX 2 selective inhibitor comprises celecoxib, it is preferred that the amount used is within a range of from about 1 to about 20 mg/day.kg, even more preferably from about 1.4 to about 8.6 mg/day.kg, and yet more preferably from about 2 to about 3 mg/day.kg.

When the COX 2 selective inhibitor comprises valdecoxib, it is preferred that the amount used is within a range of from about 0.1 to about 5 mg/day.kg, and even more preferably from about 0.8 to about 4 mg/day.kg.

When the COX 2 selective inhibitor comprises parecoxib, it is preferred that the amount used is within a range of from about 0.1 to about 5 mg/day.kg, and even more preferably from about 1 to about 3 mg/day.kg.

In terms of absolute daily dosages, when the COX 2 selective inhibitor comprises rofecoxib, it is preferred that the amount used is from about 10 to about 75 mg/day, more preferably from about 12.5 to about 50 mg/day. When the COX 2 selective inhibitor comprises etoricoxib, it is preferred that the amount used is from about 50 to about 100 mg/day, more preferably from about 60 to about 90 mg/day. When the COX 2 selective inhibitor comprises celecoxib, it is preferred that the amount used is from about 100 to about 1000 mg/day, more preferably from about 200 to about 800 mg/day. When the COX 2 selective inhibitor comprises valdecoxib, it is preferred that the amount used is from about 5 to about 100 mg/day, more preferably from about 10 to about 60 mg/day. When the COX 2 selective inhibitor comprises parecoxib, it is preferred that the amount used is within a range of from about 10 to about 100 mg/day, more preferably from about 20 to about 80 mg/day.

The COX 2 selective inhibitor(s) and second drugs that are described above can be provided in a therapeutic composition so that the preferred amounts thereof is/are supplied by a single dosage, a single capsule for example, or, by up to four, or more, single dosage forms.

In one embodiment of the invention, the COX 2 inhibitor(s) and the second drug(s) may be administered substantially simultaneously, meaning that both agents may be provided in a single dosage, for example by mixing the agents and incorporating the mixture into a single capsule. Otherwise, the COX 2 inhibitor(s) and second drug(s) may be administered substantially simultaneously by administration in separate dosages within a short time period, for example within 5 minutes or less. Alternatively, the COX 2 inhibitor(s) and second drug(s) may be administered sequentially, meaning that separate dosages, and possibly even separate dosage forms of the COX 2 inhibitor(s) and second drugs(s) may be administered at separate times, for example on a staggered schedule but with equal frequency of administration of the COX 2 inhibitor(s) and the second drug(s). Of course, it is also possible that the COX 2 inhibitor(s) may be administered either more or less frequently than the second drug(s). In any case, it is preferable that, among successive time periods of a sufficient length, for example one day, the weight ratio of the COX 2 inhibitor(s) administered to the weight ratio of the second drug(s) administered remains constant.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in connection with use of the method(s) of the present invention are the isomeric forms and tautomers and the pharmaceutically-acceptable salts of the cyclooxygenase-2 selective inhibitors and the second drugs. Isomers of COX 2 inhibitors and second drugs include their diastereomers, enantiomers, and racemates as well as their structural isomers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric, and galacturonic acids.

Suitable pharmaceutically-acceptable base addition salts of compounds used in connection with the method(s) of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention. Pharmaceutically acceptable esters include, but are not limited to, the alkyl esters of both the COX 2 inhibitors and the second drugs. For example, the second drug levodopa may be administered as its methyl ester or its ethyl ester.

The method of the present invention is useful for, but not limited to, the prevention, inhibition, and/or treatment of PD.

As used herein, the terms “PD” and “cyclooxygenase-2 mediated disorder” are meant to include, without limitation, each of the symptoms associated with Parkinson's disease that is mentioned in this application.

The present method includes the treatment, inhibition and/or prevention of a cyclooxygenase-2 mediated disorder in a subject, where the method comprises treating the subject having or susceptible to the disorder with a combined therapeutically-effective amount of the cyclooxygenase-2 selective inhibitor(s) and second drug(s) that are described in this specification. This method is useful where the cyclooxygenase-2 mediated disorder is PD.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of undesirable symptoms associated with PD. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has pain, inflammation and/or any one of the known inflammation-associated disorders. The subject is typically a human subject.

For methods of prevention, the subject is any human or animal subject, and preferably is a subject that is in need of prevention and/or treatment of PD. The subject may be a human subject who is at risk for PD. The subject may be at risk for PD due to genetic predisposition, lifestyle, diet, exposure to disorder-causing agents, exposure to pathogenic agents and the like.

In connection with the inventive method, the COX 2 pharmaceutical composition(s) and second drug(s) may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrase “administration” in defining the use of both a cyclooxygenase-2 inhibitor agent and a second drug is intended to embrace administration of each agent in a manner and in a regimen that will provide beneficial effects of the drug combination therapy, and is intended as well to embrace co-administration of 2 or more of the COX 2 agents in a substantially simultaneous manner and/or 2 or more of the second drugs in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from the constituent COX 2 agent and second drug used in combination. For example, when the treatment of PD in accordance with the present invention comprises administering a COX 2 enzyme inhibitor in combination with levodopa, it may be preferable to use levodopa in combination with a decarboxylase enzyme inhibitor such as carbidopa or benserazide.

The phrases “therapeutically-effective” and “effective for the treatment, prevention, or inhibition”, are intended to qualify the amount of each COX 2 agent and each second drug for use in the COX 2 therapy which will achieve the goal of reduction of the severity and/or frequency of incidence of PD associated symptoms, while avoiding adverse side effects typically associated with alternative therapies.

In particular, the pharmaceutical composition of one or more COX 2 inhibitors and one or more second drugs in connection with the method(s) of the present invention can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject pharmaceutical composition of COX 2 inhibitor(s) and second drug(s) in connection with the present inventive method can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject pharmaceutical composition of COX 2 inhibitor(s) and second drug(s) in connection with the present inventive method can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The pharmaceutical compositions of COX 2 inhibitor(s) and second drug(s) in connection with the present inventive method can also be administered topically, in the form of patches, creams, ointments, jellies, collyriums, solutions or suspensions. Of course, the compositions of the present invention can be administered by routes of administration other than topical administration. Also, as mentioned above, the COX 2 inhibitor(s) and second drug(s) may be administered separately, with each agent administered by any of the above mentioned administration routes. For example, the COX 2 inhibitor(s) may be administered orally in any or the above mentioned forms (e.g. in capsule form) while the second drug(s) is/are administered topically (e.g. as a cream).

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

The following examples describe embodiments of the invention. Other embodiments within the scope of the embodiments herein will be apparent to one skilled in the art from consideration of the specification or practice of the invention as disclosed herein. It is intended that the specification, together with the examples, be considered to be exemplary only, with the scope and spirit of the invention being indicated by the embodiments and the examples. In the examples, all percentages are given on a weight basis unless otherwise indicated.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that several advantages of the invention are achieved and other advantageous results obtained.

As various changes could be made in the above methods and compositions without departing from the scope of the invention, it is intended that all matter contained in this application shall be interpreted as illustrative and not in a limiting sense. Exemplary non-limiting embodiments of the present invention are provided below.

Exemplary PD symptoms that may be treated with the compositions of Tables 1-1A above are indicated in Table 3 below:

TABLE 3

| | Exemplary PD Indication(s) treated with the COX 2-specific |
|---|---|
| No. | inhibitor of Tables 1-1A |
| 1. | Tremor |
| 2. | Rigidity |
| 3. | Bradykinesia |
| 4. | Postural defects |
| 5. | Reduced blinking |
| 6. | Difficulties in communicating including, but not limited to, voice |
| | volume and tone, etc. |
| 7. | Micrographia |
| 8. | Impaired ocular conversion |
| 9. | Sialorrhea |
| 10. | Seborrhea |
| 11. | Loss of facial expression |
| 12. | Freezing |
| 13. | Depression |
| 14. | Hallucinations |
| 15. | Psychiatric Manifestations |

The following Tables 4 and 5 list various dosage forms of the pharmaceutical composition for use in conjunction with the method of the present invention. Note that the dosage forms in Table 5 exclude all dosage forms that may be transdermally applied. By contrast, Table 6 includes such transdermally applied dosage forms.

TABLE 4

| | Exemplary Dosage Forms (other than those that are transdermally |
|---|---|
| No. | applied) |
| | Oral dosage forms |
| 1. | Tablet |
| 2. | Slow Release Tablet |
| 3. | Effervescent Tablet |
| 4. | Enteric Coated Tablet |
| 5. | Compressed Tablet |
| 6. | Molded Tablet |
| 7. | Capsule |
| 8. | Slow Release Capsule |
| 9. | Capsule for Use in or with Nebulizer |
| 10. | Gelatin Capsule |
| 11. | Caplet |
| 12. | Troche |
| 13. | Powder |
| 14. | Lozenge |
| 15. | Gum |
| 16. | Solution |
| 17. | Suspension |
| 18. | Emulsion |
| 19. | Dispersion |
| | Parenteral Dosage Forms |
| 20. | Intramuscular Injection |
| 21. | Intravenous Injection |
| 22. | Inhalant |
| 23. | Aerosol |
| 24. | Nebulizing Liquid |
| 25. | Elixir |
| 26. | Collyria |
| 27. | Injection |
| 28. | Pellets |
| 29. | Implants |
| 30. | Otic Solution |
| 31. | Suppository |
| 32. | Syrup |
| 33. | Tincture |
| 34. | Opthalmic Solution |
| 35. | Oral Gel |
| 36. | Oral Paste |
| 37. | Oral Inhalant |

TABLE 5

| No. | Exemplary dosage Forms (that are topically applied) |
|---|---|
| 1. | Liquid |
| 2. | Emulsion |
| 3. | Dispersion |
| 4. | Gel |
| 5. | Paste |
| 6. | Cream |
| 7. | Lotion |
| 8. | Extract |
| 9. | Ointment |
| 10. | Patch |
| 11. | Implant |
| 12. | Pellet |
| 13. | Topical Powder |
| 14. | Topical Solution |

For a more complete list of dosage forms in addition to those provided in Tables 4 and 5, see Remington's Pharmaceutical Sciences, Mack Publishing Co., Easton, Pa., Arthur Osol (editor), 16^{th} Edition (1980). Also see each of the later editions of the same (i.e., each later edition to date of Remington's Pharmaceutical Sciences). Also see, The United States Pharmacopeia, 21^{st} Edition, United States Pharmacopeial Convention, Washington, D.C. (1985). Also see each of the later editions of the same (i.e., each later edition to date of The United States Pharmacopeia).

1. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (I): 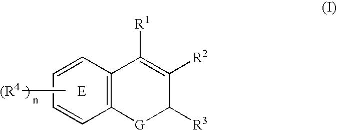
or an isomer, a pharmaceutically acceptable salt, ester, or prodrug thereof;
wherein:
G is O, S or NR^{a};
R^{a} is alkyl;
R¹ is H or aryl;
R² is carboxyl, aminocarbonyl, alkylsulfonylaminocarbonyl or alkoxycarbonyl;
R³ is haloalkyl, alkyl, aralkyl, cycloalkyl or aryl optionally and independently substituted with one or more radicals selected from alkylthio, nitro and alkylsulfonyl;
n is an integer which is 1, 2, 3, or 4; and
each R⁴ is independently H, halo, alkyl, aryl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, mono- or dialkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, aralkylcarbonyl, heteroarylcarbonyl, arylcarbonyl, aminocarbonyl, alkylcarbonyl, aryl, or heteroaryl;
wherein said aryl and heteroaryl radicals are optionally and independently substituted with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
or wherein R⁴ together with the atoms to which R⁴ is attached and the remainder of ring E forms a naphthyl radical.

2. The method of claim 1 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

3. The method of claim 1 wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, or a selective serotonin reuptake inhibitor.

4. The method of claim 1, wherein:
G is O or S ;
R² is carboxyl, lower alkyl, lower aralkyl and lower alkoxycarbonyl;
R³ is lower haloalkyl, lower cycloalkyl and phenyl; and
each of one or more R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, lower aralkylcarbonyl, lower alkylcarbonyl, and phenyl optionally and independently substituted with one or more radicals selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
or wherein R⁴ together with the atoms to which R⁴ is attached and the remainder of ring E forms a naphthyl radical.

5. The method of claim 4, wherein:
R² is carboxyl;
R³ is lower haloalkyl; and
each of one or more R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl;
or wherein R⁴ together with the atoms to which R⁴ is attached and the remainder of ring E forms a naphthyl radical.

6. The method of claim 5, wherein:
said lower haloalkyl R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each or one or more R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, benzylaminosulfonyl, N-ethylsulfonyl, 2,2-dimethylethylaminosulfonyl, N,N-dimethylaminosulfonyl, isopropylaminosulfonyl, N-(2-methylpropyl)aminosulfonyl, N-morpholinosulfonyl, methylsulfonyl, benzylcarbonyl, 2,2-dimethylpropylcarbonyl, phenylacetyl, or phenyl;
or wherein R⁴ together with the atoms to which R⁴ is attached and the remainder of the ring E forms a naphthyl radical.

7. The method of claim 6, wherein:
R³is trifluoromethyl or pentafluoroethyl; and
each of one or more R⁴ is independently H, chloro, fluoro, bromo, iodo, methyl, ethyl, isopropyl, tert-butyl, methoxy, trifluoromethyl, trifluoromethoxy, N,N-diethylamino, N-phenylmethylaminosulfonyl, N-phenylethylaminosulfonyl, N-(2-furylmethyl)aminosulfonyl, N,N-dimethylaminosulfonyl, N-methylaminosulfonyl, benzylaminosulfonyl, N-(2,2-dimethylethyl)aminosulfonyl, isopropylaminosulfonyl, dimethylaminosulfonyl, 2-methylpropylaminosulfonyl, N-morpholinosulfonyl, methylsulfonyl, benzylcarbonyl, or phenyl;
or wherein R⁴ together with the atoms to which R⁴ is attached and the remainder of ring E forms a naphthyl radical.

8. The method of claim 7, wherein:
R³ is trifluoromethyl or pentafluoroethyl;
each of one or more R⁴ is independently H, methyl, ethyl, isopropyl, tert-butyl, chloro, bromo, fluoro, iodo, methyl, tert-butyl, trifluoromethoxy, methoxy, benzylcarbonyl, dimethylaminosulfonyl, isopropylaminosulfonyl, N-methylaminosulfonyl, benzylaminosulfonyl, phenylethylaminosulfonyl, methylpropylaminosulfonyl, methylsulfonyl, morpholinosulfonyl, N,N-diethylamino, or phenyl.

9. The method of claim 1, wherein said COX 2 inhibitor or isomer, pharmaceutically acceptable salt, ester, or prodrug thereof has a COX 2 IC₅₀ of less than about 5 μmol/L.

10. The method of claim 1, wherein said COX 2 inhibitor or isomer, pharmaceutically acceptable salt, ester, or prodrug thereof has a selectivity ratio of COX 1 IC₅₀ to COX 2 IC₅₀ of at least about 10.

11. The method of claim 10, wherein said COX 2 inhibitor or isomer, pharmceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

12. The method of claim 1, wherein said COX 2 inhibitor or isomer, pharmaceutically acceptable salt, ester, or prodrug thereof has a COX 1 IC₅₀ of at least about 1 μmol/L.

13. The method of claim 12, wherein said COX 2 inhibitor or isomer, pharmaceutically acceptable salt, ester, or prodrug thereof has a COX 1 IC₅₀ of at least about 20 μmol/L.

14. The method of claim 1, wherein said first amount is from about 0.001 to about 100 mg/day per kg of body weight of said subject and said second amount is from about 0.001 to about 100 mg/day per kg of body weight of said subject.

15. The method of claim 14, wherein said first amount is from about 0.5 to about 50 mg/day per kg of body weight of said subject and said second amount is from about 0.1 to about 10 mg/day per kg of body weight of said subject.

16. The method of claim 15, wherein said first amount is from about 1 to about 20 mg/day per kg of body weight of said subject and said second amount is from about 0.5 to about 2 mg/day per kg of body weight of said subject.

17. The method of claim 1 wherein a weight ratio of said first amount to said second amount is from about 0.002 to about 10.

18. The method of claim 17 wherein a weight ratio of said first amount to said second amount is from about 0.1 to about 5.

19. The method of claim 1, wherein said subject is an animal.

20. The method of claim 19, wherein said subject is a human.

21. The method of claim 1, wherein said COX 2 inhibitor or isomer, pharmaceutically acceptable salt, ester, or prodrug thereof and said second drug are administered enterally or parenterally in one or more doses per day.

22. The method of claim 1, wherein said COX 2 inhibitor or isomer, pharmaceutically acceptable salt, ester, or prodrug thereof and said second drug are administered substantially simultaneously.

23. The method of claim 1, wherein said COX 2 inhibitor or isomer, pharmaceutically acceptable salt, ester, or prodrug thereof and said second drug are administered sequentially.

24. A method for treating Parkinson's disease comprising administering, to a subject in need thereof, a cyclooxygenase-2 (COX 2) inhibitor in a first amount and a second drug in a second amount, wherein said first amount together with said second amount is a therapeutically effective amount of said COX 2 inhibitor and said second drug, and wherein said COX 2 inhibitor is represented by Formula (II): 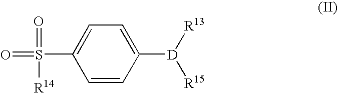
or an isomer, a pharmaceutically acceptable salt, ester, or prodrug thereof,
provided that Formula (II) is not celecoxib (B-18) or rofecoxib (B-21),
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

25. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (II): 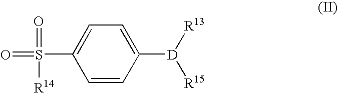
or an isomer, a pharmaceutically acceptable salt, ester, or prodrug thereof,
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,
wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, pharmaceutically acceptable salt, ester, or prodrug thereof.

26. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (II): 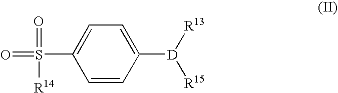
or an isomer, a pharmaceutically acceptable salt, ester, or prodrug thereof,
wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl; and,
wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

27. The method of claim 24, wherein said COX 2 inhibitor or isomer, pharmaceutically acceptable salt, ester, or prodrug thereof has a COX 2 IC₅₀ of less than about 5 μmol/L.

28. The method of claim 24, wherein said COX 2 inhibitor or isomer, pharmaceutically acceptable salt, ester, or prodrug thereof has a selectivity ratio of COX 1 IC₅₀ to COX 2 IC₅₀ of at least about 10.

29. The method of claim 28, wherein said COX 2 inhibitor or isomer, pharmceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

30. The method of claim 24, wherein said COX 2 inhibitor or isomer, pharmaceutically acceptable salt, ester, or prodrug thereof has a COX 1 IC₅₀ of at least about 1μmol/L.

31. The method of claim 30, wherein said COX 2 inhibitor or isomer, pharmaceutically acceptable salt, ester, or prodrug thereof has a COX 1 IC₅₀ of at least about 20 μmol/L.

32. The method of claim 24, wherein said first amount is from about 0.001 to about 100 mg/day per kg of body weight of said subject and said second amount is from about 0.001 to about 100 mg/day per kg of body weight of said subject.

33. The method of claim 32, wherein said first amount is from about 0.5 to about 50 mg/day per kg of body weight of said subject and said second amount is from about 0.1 to about 10 mg/day per kg of body weight of said subject.

34. The method of claim 33, wherein said first amount is from about 1 to about 20 mg/day per kg of body weight of said subject and said second amount is from about 0.5 to about 2 mg/day per kg of body weight of said subject.

35. The method of claim 24 wherein a weight ratio of said first amount to said second amount is from about 0.002 to about 10.

36. The method of claim 24 wherein a weight ratio of said first amount to said second amount is from about 0.1 to about 5.

37. The method of claim 24, wherein said subject is an animal.

38. The method of claim 37, wherein said subject is a human.

39. The method of claim 24, wherein said COX 2 inhibitor or isomer, pharmaceutically acceptable salt, ester, or prodrug thereof and said second drug are administered enterally or parenterally in one or more doses per day.

40. The method of claim 24, wherein said COX 2 inhibitor or isomer, pharmaceutically acceptable salt, ester, or prodrug thereof and said second drug are administered substantially simultaneously.

41. The method of claim 24, wherein said COX 2 inhibitor or isomer, pharmaceutically acceptable salt, ester, or prodrug thereof and said second drug are administered sequentially.

42. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (III): 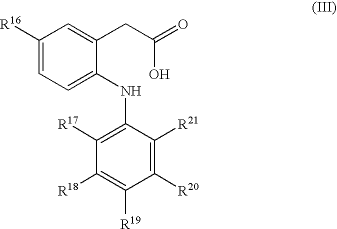
or an isomer, a pharmaceutically acceptable salt, ester, or prodrug thereof, wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

43. The method of claim 42 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

44. The method of claim 42 wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

45. The method of claim 42, wherein: R¹⁶ is ethyl; R¹⁷ and R¹⁹ are chloro; R¹⁸ and R²⁰ are hydrogen; and R²¹ is methyl.

46. The method of claim 42, wherein said COX 2 inhibitor or isomer, pharmaceutically acceptable salt, ester, or prodrug thereof has a COX 2 IC₅₀ of less than about 5 μmol/L.

47. The method of claim 42, wherein said COX 2 inhibitor or isomer, pharmaceutically acceptable salt, ester, or prodrug thereof has a selectivity ratio of COX 1 IC₅₀ to COX 2 IC₅₀ of at least about 10.

48. The method of claim 47, wherein said COX 2 inhibitor or isomer, pharmceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

49. The method of claim 42, wherein said COX 2 inhibitor or isomer, pharmaceutically acceptable salt, ester, or prodrug thereof has a COX 1 IC₅₀ of at least about 1 μmol/L.

50. The method of claim 49, wherein said COX 2 inhibitor or isomer, pharmaceutically acceptable salt, ester, or prodrug thereof has a COX 1 IC₅₀ of at least about 20 μmol/L.

51. The method of claim 42, wherein said first amount is from about 0.001 to about 100 mg/day per kg of body weight of said subject and said second amount is from about 0.001 to about 100 mg/day per kg of body weight of said subject.

52. The method of claim 51, wherein said first amount is from about 0.5 to about 50 mg/day per kg of body weight of said subject and said second amount is from about 0.1 to about 10 mg/day per kg of body weight of said subject.

53. The method of claim 52, wherein said first amount is from about 1 to about 20 mg/day per kg of body weight of said subject and said second amount is from about 0.5 to about 2 mg/day per kg of body weight of said subject.

54. The method of claim 42 wherein a weight ratio of said first amount to said second amount is from about 0.002 to about 10.

55. The method of claim 54 wherein a weight ratio of said first amount to said second amount is from about 0.1 to about 5.

56. The method of claim 42, wherein said subject is an animal.

57. The method of claim 56, wherein said subject is a human.

58. The method of claim 42, wherein said COX 2 inhibitor or isomer, pharmaceutically acceptable salt, ester, or prodrug thereof and said second drug are administered enterally or parenterally in one or more doses per day.

59. The method of claim 42, wherein said COX 2 inhibitor or isomer, pharmaceutically acceptable salt, ester, or prodrug thereof and said second drug are administered substantially simultaneously.

60. The method of claim 42, wherein said COX 2 inhibitor or isomer, pharmaceutically acceptable salt, ester, or prodrug thereof and said second drug are administered sequentially.

61. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (IV): 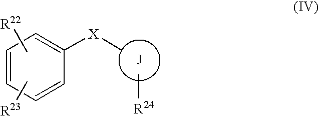
or an isomer, a pharmaceutically acceptable salt, ester, or prodrug thereof, wherein:
X is 0 or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

62. The method of claim 61 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

63. The method of claim 62 wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

64. The method of claim 61 wherein said COX 2 inhibitor is nimesulide (B-212), flosulide (B-213), NS-398 (B-26), L-745337 (B-214), RWJ-63556 (B-215), or L-784512 (B-216).

65. The method of claim 61, wherein said COX 2 inhibitor or isomer, pharmaceutically acceptable salt, ester, or prodrug thereof has a COX 2 IC₅₀ of less than about 5 μmol/L.

66. The method of claim 61, wherein said COX 2 inhibitor or isomer, pharmaceutically acceptable salt, ester, or prodrug thereof has a selectivity ratio of COX 1 IC₅₀ to COX 2 IC₅₀ of at least about 10.

67. The method of claim 66, wherein said COX 2 inhibitor or isomer, pharmceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

68. The method of claim 61, wherein said COX 2 inhibitor or isomer, pharmaceutically acceptable salt, ester, or prodrug thereof has a COX 1 IC₅₀ of at least about 1 μmol/L.

69. The method of claim 68, wherein said COX 2 inhibitor or isomer, pharmaceutically acceptable salt, ester, or prodrug thereof has a COX 1 IC₅₀ of at least about 20 μmol/L.

70. The method of claim 61, wherein said first amount is from about 0.001 to about 100 mg/day per kg of body weight of said subject and said second amount is from about 0.001 to about 100 mg/day per kg of body weight of said subject.

71. The method of claim 70, wherein said first amount is from about 0.5 to about 50 mg/day per kg of body weight of said subject and said second amount is from about 0.1 to about 10 mg/day per kg of body weight of said subject.

72. The method of claim 71, wherein said first amount is from about 1 to about 20 mg/day per kg of body weight of said subject and said second amount is from about 0.5 to about 2 mg/day per kg of body weight of said subject.

73. The method of claim 61 wherein a weight ratio of said first amount to said second amount is from about 0.002 to about 10.

74. The method of claim 73 wherein a weight ratio of said first amount to said second amount is from about 0.1 to about 5.

75. The method of claim 61, wherein said subject is an animal.

76. The method of claim 75, wherein said subject is a human.

77. The method of claim 61, wherein said COX 2 inhibitor or isomer, pharmaceutically acceptable salt, ester, or prodrug thereof and said second drug are administered enterally or parenterally in one or more doses per day.

78. The method of claim 61, wherein said COX 2 inhibitor or isomer, pharmaceutically acceptable salt, ester, or prodrug thereof and said second drug are administered substantially simultaneously.

79. The method of claim 61, wherein said COX 2 inhibitor or isomer, pharmaceutically acceptable salt, ester, or prodrug thereof and said second drug are administered sequentially.

80. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor is represented by Formula (V): 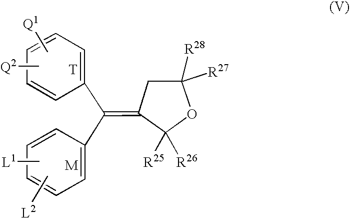
or an isomer, pharmaceutically acceptable salt, ester, or prodrug thereof, wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms, a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

81. The method of claim 80 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

82. The method of claim 80 wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

83. The method of claim 80 wherein said COX 2 inhibitor is N-(2-cyclohexyloxynitrophenyl)methane sulfonamide, or (E)-4-[(4-methylphenyl)(tetrahydro-2-oxo-3-furanylidene) methyl] benzenesulfonamide.

84. The method of claim 80, wherein said COX 2 inhibitor or isomer, pharmaceutically acceptable salt, ester, or prodrug thereof has a COX 2 IC₅₀ of less than about 5 μmol/L.

85. The method of claim 80, wherein said COX 2 inhibitor or isomer, pharmaceutically acceptable salt, ester, or prodrug thereof has a selectivity ratio of COX 1 IC₅₀ to COX 2 IC₅₀ of at least about 10.

86. The method of claim 85, wherein said COX 2 inhibitor or isomer, pharmceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

87. The method of claim 80, wherein said COX 2 inhibitor or isomer, pharmaceutically acceptable salt, ester, or prodrug thereof has a COX 1 IC₅₀ of at least about 1 μmol/L.

88. The method of claim 87, wherein said COX 2 inhibitor or isomer, pharmaceutically acceptable salt, ester, or prodrug thereof has a COX 1 IC₅₀ of at least about 20 μmol/L.

89. The method of claim 80, wherein said first amount is from about 0.001 to about 100 mg/day per kg of body weight of said subject and said second amount is from about 0.001 to about 100 mg/day per kg of body weight of said subject.

90. The method of claim 89, wherein said first amount is from about 0.5 to about 50 mg/day per kg of body weight of said subject and said second amount is from about 0.1 to about 10 mg/day per kg of body weight of said subject.

91. The method of claim 90, wherein said first amount is from about 1 to about 20 mg/day per kg of body weight of said subject and said second amount is from about 0.5 to about 2 mg/day per kg of body weight of said subject.

92. The method of claim 80 wherein a weight ratio of said first amount to said second amount is from about 0.002 to about 10.

93. The method of claim 92 wherein a weight ratio of said first amount to said second amount is from about 0.1 to about 5.

94. The method of claim 80, wherein said subject is an animal.

95. The method of claim 94, wherein said subject is a human.

96. The method of claim 80, wherein said COX 2 inhibitor or isomer, pharmaceutically acceptable salt, ester, or prodrug thereof and said second drug are administered enterally or parenterally in one or more doses per day.

97. The method of claim 80, wherein said COX 2 inhibitor or isomer, pharmaceutically acceptable salt, ester, or prodrug thereof and said second drug are administered substantially simultaneously.

98. The method of claim 80, wherein said COX 2 inhibitor or isomer, pharmaceutically acceptable salt, ester, or prodrug thereof and said second drug are administered sequentially.

99. A method for treating Parkinson's disease comprising administering to a subject in need thereof a COX 2 inhibitor in a first amount and a second drug in a second amount, wherein said first amount together with said second amount is a therapeutically effective amount of said COX 2 inhibitor and said second drug, and wherein said COX 2 inhibitor comprises B-1, B-2, B-3, B-4, B-5, B-6, B-7, B-8, B-9, B-10, B-11, B-12, B-13, B-14, B-15, B-16, B-17, B-18, B-19, B-20, B-21, B-22, B-23, B-24, B-25, B-26, B-27, B-28, B-29, B-30, B-31, B-32, B-33, B-34, B-35, B-36, B-37, B-38, B-39, B-40, B-41, B-42, B-43, B-44, B-45, B-46, B-47, B-48, B-49, B-50, B-51, B-52, B-53, B-54, B-55, B-56, B-57, B-58, B-59, B-60, B-61, B-62, B-63, B-64, B-65, B-66, B-67, B-68, B-69, B-70, B-71, B-72, B-73, B-74, B-75, B-76, B-77, B-78, B-79, B-80, B-81, B-82, B-83, B-84, B-85, B-86, B-87, B-88, B-89, B-90, B-91, B-92, B-93, B-94, B-95, B-96, B-97, B-98, B-99, B-100, B-101, B-102, B-103, B-104, B-105, B-106, B-107, B-108, B-109, B-110, B-111, B-112, B-113, B-114, B-115, B-116, B-117, B-118, B-119, B-120, B-121, B-122, B-123, B-124, B-125, B-126, B-127, B-128, B-129, B-130, B-131, B-132, B-133, B-134, B-135, B-136, B-137, B-138, B-139, B-140, B-141, B-142, B-143, B-144, B-145, B-146, B-147, B-148, B-149, B-150, B-151, B-152, B-153, B-154, B-155, B-156, B-157, B-158, B-159, B-160, B-161, B-162, B-163, B-164, B-165, B-166, B-167, B-168, B-169, B-170, B-171, B-172, B-173, B-174, B-175, B-176, B-177, B-178, B-179, B-180, B-181, B-182, B-183, B-184, B-185, B-186, B-187, B-188, B-189, B-190, B-191, B-192, B-193, B-194, B-195, B-196, B-197, B-198, B-199, B-200, B-201, B-202, B-203, B-204, B-205, B-206, B-207, B-208, B-209, B-210, B-211, B-212, B-213, B-214, B-215, B-216, B-217, B-218, B-219, B-220, B-221, B-222, B-223, B-224, B-225, B-226, B-227, B-228, B-229, B-230, B-231, B-232, or an isomer, a pharmaceutically acceptable salt, ester, or prodrug thereof; and,

100. The method of claim 99 wherein said second drug comprises C-1, C-2, C-3, C-4, C-5, C-6, C-7, C-8, C-9, C-10, C-11, C-12, C-13, C-14, C-15, C-16, C-17, C-18, C-19, C-20, C-21, C-22, C-23, C-24, C-25, C-26, C-27, C-28, C-29, C-30, C-31, C-32, C-33, C-34, C-35, C-36, C-37, C-38, C-39, C-40, C-41, C-42, C-43, C-44, C-45, C-46, C-47, C-48, C-49, C-50, C-51, C-52, C-53, C-54, C-55, C-56, C-57, C-58, C-59, C-60, C-61, C-62, C-63, C-64, C-65, C-66, C-67, C-68, C-69, C-70, C-71, C-72, C-73, C-74, C-75, C-76, C-77, C-78, C-79, C-80, C-81, C-82, or an isomer, a pharmaceutically acceptable salt, ester, or prodrug thereof.

101. The method of claim 99 wherein said second drug is a neurotrophic factor, dopaminergic, dopamine agonist, neuronal nicotinic receptor agonist, k opioid receptor agonist, adenosine antagonist, glutamate antagonist, dopamine transport inhibitor, anticholinergic, enzyme inhibitor, atypical neuroletic, selective serotonin reuptake inhibitor, or a drug offsetting one or more side effects of PD medications.

102. The method of claim 99 wherein said COX 2 inhibitor is celecoxib (B-18), valdecoxib (B-19), deracoxib (B-20), rofecoxib (B-21), etoricoxib (B-22), JTE-522 (B-23), parecoxib (B-24), ABT-963 (13-25), or BMS-347070 (B-74), and an isomer, a pharmaceutically acceptable salt, ester, or prodrug thereof.

103. The method of claim 102 wherein said COX 2 inhibitor is celecoxib (B-18), rofecoxib (B-21), etoricoxib (B-22), JTE-522 (B-23), parecoxib (B-24), ABT-963 (B-25), or BMS-347070 (B-74).

104. The method of claim 103, wherein said COX 2 inhibitor is sodium parecoxib.

105. The method of claim 99, wherein said COX 2 inhibitor, isomer, pharmaceutically acceptable salt, ester, or prodrug thereof has a COX 2 IC₅₀ of less than about 5 μmol/L.

106. The method of claim 99, wherein said COX 2 inhibitor, isomer, pharmaceutically acceptable salt, ester, or prodrug thereof has a selectivity ratio of COX 1 IC₅₀ to COX 2 IC₅₀ of at least about 10.

107. The method of claim 106, wherein said COX 2 inhibitor, isomer, pharmaceutically acceptable salt, ester, or prodrug thereof has a COX 2 IC₅₀ of less than about 1 μmol/L and a selectivity ratio of COX 1 IC₅₀ to COX 2 IC₅₀ of at least about 100.

108. The method of claim 99, wherein said COX 2 inhibitor, isomer, pharmaceutically acceptable salt, ester, or prodrug thereof has a COX 1 IC₅₀ of at least about 1 μmol/L.

109. The method of claim 108, wherein said COX 2 inhibitor, isomer, pharmaceutically acceptable salt, ester, or prodrug thereof has a COX 1 IC₅₀ of at least about 20 μmol/L.

110. The method of claim 99, wherein said first amount is from about 0.001 to about 100 mg/day per kg of body weight of said subject and said second amount is from about 0.001 to about 100 mg/day per kg of body weight of said subject.

111. The method of claim 110, wherein said first amount is from about 0.5 to about 50 mg/day per kg of body weight of said subject and said second amount is from about 0.1 to about 10 mg/day per kg of body weight of said subject.

112. The method of claim 111, wherein said first amount is from about 1 to about 20 mg/day per kg of body weight of said subject and said second amount is from about 0.5 to about 2 mg/day per kg of body weight of said subject.

113. The method of claim 99 wherein a weight ratio of said first amount to said second amount is from about 0.002 to about 10.

114. The method of claim 113 wherein a weight ratio of said first amount to said second amount is from about 0.1 to about 5.

115. The method of claim 99, wherein said subject is an animal.

116. The method of claim 115, wherein said subject is a human.

117. The method of claim 99, wherein said COX 2 inhibitor or isomer, pharmaceutically acceptable salt, ester, or prodrug thereof and said second drug are administered enterally or parenterally in one or more doses per day.

118. The method of claim 99, wherein said COX 2 inhibitor or isomer, pharmaceutically acceptable salt, ester, or prodrug thereof and said second drug are administered substantially simultaneously.

119. The method of claim 99, wherein said COX 2 inhibitor or isomer, pharmaceutically acceptable salt, ester, or prodrug thereof and said second drug are administered sequentially.
